(19) United States
(12) Patent Application Publication
(10) Pub. No.: US 2013/0150346 A1
(43) Pub. Date: Jun. 13, 2013

(54) Use of FAAH Inhibitors for Treating Parkinson's Disease and Restless Legs Syndrome

(71) Applicants: James Philip Pearson, Cambridge, MA (US); Todd G. Milne, Brookline, MA (US); Thomas Henry Johnston, Toronto (CA); Jonathan Michael Brotchie, Toronto (CA)

(72) Inventors: James Philip Pearson, Cambridge, MA (US); Todd G. Milne, Brookline, MA (US); Thomas Henry Johnston, Toronto (CA); Jonathan Michael Brotchie, Toronto (CA)

(73) Assignees: Quest Ventures Ltd., Manchester (GB); Ironwood Pharmaceutical, Inc., Cambrige, MA (US)

(21) Appl. No.: 13/520,644

(22) PCT Filed: Jan. 7, 2011
(86) PCT No.: PCT/US11/20539

§ 371 Date: Feb. 27, 2013

Related U.S. Application Data
(60) Provisional application No. 61/293,572, filed on Jan. 8, 2010.

(51) Int. Cl. A61K 31/4439; C07D 209/22; A61K 31/404; C07D 403/14; A61K 31/506; A61K 31/223; A61K 31/195; A61K 31/165; A61K 31/26; A61K 31/12; A61K 31/4985; A61K 31/48; A61K 31/428; A61K 31/381; A61K 31/437; A61K 31/473; A61K 31/55; A61K 31/137; A61K 31/4458; A61K 31/136; A61K 31/16; C07D 401/12

(52) U.S. Cl. CPC A61K 31/4439; C07D 401/12; C07D 209/22; A61K 31/404; C07D 403/14; A61K 31/506; A61K 31/223; A61K 31/195; A61K 31/165; A61K 31/26; A61K 31/12; A61K 31/4985; A61K 31/48; A61K 31/428; A61K 31/381; A61K 31/437; A61K 31/473; A61K 31/55; A61K 31/137; A61K 31/4458; A61K 31/136; A61K 31/16
USPC 514/215; 514/343; 514/419; 514/274; 514/269; 514/538; 514/567; 514/614; 514/521; 514/676; 514/250; 514/284; 514/367; 514/418; 514/438; 514/288; 514/649; 514/317; 514/657; 514/654; 514/620

(57) ABSTRACT

The present disclosure relates to methods of using fatty acid amide hydrolase (FAAH) inhibitors to treat aspects of Parkinson's disease (PD), restless legs syndrome (RLS) and periodic limb movement disorder (PLMD), the use of FAAH inhibitors for the manufacture of medicaments for use in the treatment of PD, RLS and PLMD, as well as pharmaceutically acceptable compositions comprising FAAH inhibitors for use in the treatment of PD, RLS and PLMD.

### PRIORITY CLAIM

This application claims priority to U.S. Provisional Application Ser. No. 61/293,572, filed on Jan. 8, 2010. The entire contents of the aforementioned application are herein incorporated by reference.

### TECHNICAL FIELD

The present disclosure relates to methods of using fatty acid amide hydrolase (FAAH) inhibitors to treat aspects of Parkinson's disease (PD), restless legs syndrome (RLS) and periodic limb movement disorder (PLMD), the use of FAAH inhibitors for the manufacture of medicaments for use in the treatment of PD, RLS and PLMD, as well as pharmaceutically acceptable compositions comprising FAAH inhibitors for use in the treatment of PD, RLS and PLMD.

### BACKGROUND

Parkinson's disease (PD) is a chronic, progressive, hypokinetic disorder characterized by impaired voluntary movement. PD occurs as a result of the death of dopamine-producing neurons in the substantia nigra of the midbrain. Dopamine is a neurotransmitter that transports signals to the parts of the brain that control movement initiation and coordination. The loss of dopamine in the brain is associated with multiple primary symptoms including: tremor of the hands, arms, legs, jaw, and face; rigidity or stiffness of the limbs and trunk; bradykinesia or slowness of movement; and postural instability or impaired balance and coordination. PD afflicts more than one million persons in the United States alone, with approximately 50,000 new cases diagnosed each year. It is generally a disease of late middle age, with typical onset occurring at about age 60. About five percent of patients, however, have early-onset disease and are younger than 40 when symptoms begin.

Dopaminergic agent therapy, such as dopamine replacement therapy, remains the most efficacious symptomatic treatment for PD. However, such strategies are associated with motor complications such as dyskinesia, wearing-off and on-off fluctuations (Fox et al., 2008, Mov Disord. 23 Suppl. 3:S509-514; Poewe, 2009, Neurology, 72:S65-73). Non-motor-related complications of treatment are increasingly being recognized as further undesirable consequences of aberrant, treatment-related, dopamine receptor stimulation. For example, treatment with the dopamine precursor 3,4-dihydroxyphenylalanine (levodopa, L-DOPA) or with dopamine receptor agonists (“dopamine agonists”) can result in excessively heightened mood and psychomotor activity, including psychosis (Witjas et al., 2002, Neurology, 59:408-413; Racette et al., 2002, J Neuropsychiatry Clin. Neurosci., 14:438-442). In addition, some patients experience dopamine dysregulation syndrome (DDS), which is characterized in part by hyperactivity with compulsive, repetitive, non-goal directed motor activity (e.g., “punding”, stereotypies, “hobbyism” and “walkabouts”). Treatment with L-DOPA or dopamine agonists can also result in impulse control disorders (ICD) such as pathological gambling, excessive eating, shopping, alcohol abuse, and hypersexuality (Potenza et al., 2007, Nat. Clin. Pract. Neurol. 3:664-672; Evans et al., 2004, Mov. Disord. 19:397-405). Further, treatment with L-DOPA or dopamine agonists can also result in impaired sleeping, including restlessness, insomnia, vivid dreaming and nightmares, and may be correlated with the presence of ICD in a patient (O'Sullivan et al. 2010, J. Neural Neurosurg. Psychiatry, doj. 10.11136/jnnp.2009.186874).

Restless legs syndrome (RLS) is a neurological disorder characterized by unpleasant sensations in the legs and an uncontrollable urge to move when at rest in an effort to relieve these feelings. The most distinctive aspect of RLS is that lying down and trying to relax activates the symptoms. As a result, most people with RLS have difficulty falling asleep and staying asleep, which causes exhaustion and daytime fatigue. Some researchers estimate that RLS affects as many as 12 million Americans, but the occurrence may be higher because RLS is thought to be underdiagnosed and misdiagnosed. More than 80 percent of people with RLS also experience a more common condition known as periodic limb movement disorder (PLMD), which is characterized by involuntary leg twitching or jerking movements during sleep that occur at periodic intervals anywhere from 20-40 seconds apart. In addition, there are many people with PLMD who do not have RLS. The overall incidence of PLMD is 4% in adults, but it is more common in the elderly, especially females, with up to 11% experiencing symptoms. The symptoms cause repeated awakening and severely disrupted sleep.

Dopaminergic agents have been shown to reduce RLS and PLMD symptoms and are considered the initial treatment of choice for RLS. However, similar to PD patients, treatment of RLS patients with dopaminergic agents, such as dopamine agonists or L-DOPA, can lead to ICD and DDS (Leu-Semenescu et al., 2009, Sleep Med., 10:494-496; Abler et al., 2009, Brain, 132:2396-2402).

The usual treatment for disorders such as DDS and ICD is to reduce dopaminergic treatments; however, this often results in worsening of motor function in PD or a worsening of RLS or PLMD symptoms. Thus, additional treatment options are required to promote motor function in PD patients or alleviate RLS or PLMD symptoms in patients while ameliorating sleep disorders, DDS and/or ICD.

### SUMMARY

The present invention addresses these issues by providing methods of treating PD, RLS or PLMD while preventing or treating sleep disorders, DDS and/or ICD. In one aspect, the invention provides a method of treating or preventing a sleep disorder, ICD and/or DDS arising from the treatment of a patient suffering from PD, RLS and/or PLMD with a dopaminergic agent, comprising administering a therapeutically effective amount of a FAAH inhibitor to the patient. In another aspect, the invention provides a method of treating PD, RLS and/or PLMD in a patient while preventing or ameliorating a sleep disorder, ICD and/or DDS, comprising administering a therapeutically effective amount of a dopaminergic agent and a therapeutically effective amount of a FAAH inhibitor to the patient. In another aspect, the invention provides pharmaceutical compositions comprising a FAAH inhibitor, optionally in combination with one or more other drugs, for use in the treatment of PD, RLS or PLMD. In another aspect, the invention provides pharmaceutical compositions comprising a FAAH inhibitor and a dopaminergic agent. The invention also provides for the use of FAAH inhibitors for the manufacture of medicaments for treating or preventing a sleep disorder, ICD and/or DDS arising from the treatment of a patient suffering from PD, RLS and/or PLMD with a dopaminergic agent.

### BRIEF DESCRIPTION OF THE DRAWINGS

FIGS. 1A-1C show the effect of treatment with the FAAH inhibitor 3-(3-carbamoylphenyl)phenyl]N-cyclohexylcarbamate (URB597) on plasma FAAH substrate levels in 1-methyl-4-phenyl-1,2,3,6-tetrahydropyridine (MPTP)-lesioned marmosets. Experimental protocols are described in Example 3. Plasma levels of N-arachidonoyl-ethanolamide (AEA, anandamide; FIG. 1A), N-oleoyl-ethanolamide (OEA; FIG. 1B) and N-palmitoyl-ethanolamide (PEA; FIG. 1C) were assessed via LC-MS/MS. The number of animals was n=6 for all groups. Data are mean values with SEM; *** represents p<0.001 cf. vehicle-treated animals (RM-ANOVA with Student Newman-Keuls Multiple Comparison Test).

FIG. 2 shows the time course of total motor activity in MPTP-lesioned marmosets treated with vehicle/vehicle (open circles), URB597/vehicle (closed circles), vehicle/L-DOPA (open triangles), or URB597/L-DOPA (closed triangles). Experimental protocols are described in Example 5. Data counts are plotted as total activities/30 min interval. Asterisks (*) indicate significant differences (all p<0.05) cf. vehicle alone.

FIGS. 3A-3C show the effect of treatment with URB597 on total motor activity, high activity counts and high activity time in MPTP-lesioned marmosets compared with non-treated, normal un-lesioned marmosets, during the period 2-4 h after administration of L-DOPA, which had the highest activity (see FIG. 2). Total activity in the period 2-4 h after L-DOPA is shown in (FIG. 3A) and also further compared to that same minute obtained pre-MPTP (normal—black bar). High counts (FIG. 3B) and minutes (FIG. 3C) of high activity (i.e., above the average of that animal in the normal state) were cumulated. The number of animals was n=6 for all groups. Data are mean values with SEM. The symbols */*** represent p<0.05 or p<0.001, respectively, cf. vehicle (p.o.)/vehicle (s.c.) treated, MPTP-lesioned animals; #/## represent p<0.05 or p<0.01, respectively, cf. vehicle (p.o.)/L-DOPA (s.c.) treated, MPTP-lesioned animals; †/††† represent p<0.05/p<0.001, respectively, cf. normal (untreated pre-MPTP) animals. (RM-ANOVA with Student Newman-Keuls Multiple Comparison Test).

FIG. 4 shows the effect of treatment with URB597 on parkinsonian disability, dyskinesia and psychosis in MPTP-lesioned marmosets. Experimental protocols are described in Example 5. Scores for parkinsonian disability (FIG. 4A), dyskinesia (FIG. 4B) and psychosis (FIG. 4C) were obtained every 10 min and summed over the period of peak L-DOPA action (2-4 h). The number of animals was n=6 for all groups. Data are expressed as medians with individual values. Asterisks (*) represent p<0.05 cf. vehicle (p.o.)/vehicle (s.c.) treated animals, using Friedman's test with Dunn's multiple comparison test.

FIGS. 5A-5K provide references and structures for exemplary FAAH inhibitors.

FIG. 6 shows the time course of total motor activity in normal macaques, or MPTP-lesioned macaques treated with vehicle, L-DOPA, or URB597/L-DOPA. Experimental protocols are described in Examples 6 and 7. (*/** represent p<0.05 or p<0.01, respectively, cf. L-DOPA treatment alone in MPTP-lesioned macaques.)

FIG. 7 shows total motor activity in normal macaques, or MPTP-lesioned macaques treated with vehicle, L-DOPA, or URB597/L-DOPA, between 2 and 3 hours after onset of observation (treatment with L-DOPA). Experimental protocols are described in Examples 6 and 7. (**/*** represent p<0.01 or p<0.001, respectively, cf. vehicle (p.o.)/vehicle (s.c.) treated, MPTP-lesioned animals; # represents p<0.05 cf. vehicle (p.o.)/L-DOPA (s.c.) treated, MPTP-lesioned animals).

FIG. 8 shows the time course in high activity time in normal macaques, or MPTP-lesioned macaques treated with vehicle, L-DOPA, or URB597/L-DOPA. Experimental protocols are described in Examples 6 and 7. (* represents p<0.05 cf. L-DOPA treatment alone in MPTP-lesioned macaques.)

FIG. 9 shows high activity time in normal macaques, or MPTP-lesioned macaques treated with vehicle, L-DOPA, or URB597/L-DOPA between 2 and 3 hours after onset of observation (treatment with L-DOPA). Experimental protocols are described in Examples 6 and 7. (** represents p<0.01 cf. vehicle (p.o.)/vehicle (s.c.) treated, MPTP-lesioned animals).

FIG. 10 shows the time course in high activity counts in normal macaques, or MPTP-lesioned macaques treated with vehicle, L-DOPA, or URB597/L-DOPA. Experimental protocols are described in Examples 6 and 7. (*/*** represent p<0.05 or p<0.001, respectively, cf. L-DOPA treatment alone in MPTP-lesioned macaques.)

FIG. 11 shows high activity counts of normal macaques, or MPTP-lesioned macaques treated with vehicle, L-DOPA, or URB597/L-DOPA between 2 and 3 hours after onset of observation (treatment with L-DOPA). Experimental protocols are described in Examples 6 and 7. (** represents p<0.01 cf. vehicle (p.o.)/vehicle (s.c.) treated, MPTP-lesioned animals; # represents p<0.05 cf. vehicle (p.o.) 1 L-DOPA (s.c.) treated, MPTP-lesioned animals).

The figures are provided by way of examples and are not intended to limit the scope of the present invention.

### DETAILED DESCRIPTION

Reference will now be made in detail to certain embodiments of the invention, examples of which are illustrated in the accompanying structures and formulae. While the invention will be described in conjunction with the enumerated embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and materials described herein but include any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls.

### DEFINITIONS

The compounds described herein may be defined by their chemical structures and/or chemical names. Where a compound is referred to by both a chemical structure and a chemical name, and the chemical structure and chemical name conflict, the chemical structure is determinative of the compound's identity.

The term “halo” or “halogen” refers to any radical of fluorine, chlorine, bromine or iodine.

As used herein, the term “cyano” refers to —CN or —C≡N.

The term “hydroxyl” or “hydroxy” refers to —OH.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it (i.e., 1, 2, 3, 4, 5, 6, 7, 8, 9, 10, 11, 12). The term “haloalkyl” refers to an alkyl in which one or more hydrogen atoms are replaced by halo, and includes alkyl moieties in which all hydrogens have been replaced by halo (e.g., perfluoroalkyl). The terms “arylalkyl” or “aralkyl” refer to an alkyl moiety in which an alkyl hydrogen atom is replaced by an aryl group. Examples of “arylalkyl” or “aralkyl” include, but are not limited to, benzyl and 9-fluorenyl groups.

The term “alkenyl” refers to a linear or branched-chain monovalent hydrocarbon radical with at least one site of unsaturation, i.e., a carbon-carbon, sp² double bond, wherein the alkenyl radical includes radicals having “cis” and “trans” orientations, or alternatively, “E” and “Z” orientations. Unless otherwise specified, an alkenyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, vinyl, allyl and the like.

The term “alkynyl” refers to a linear or branched monovalent hydrocarbon radical with at least one site of unsaturation, i.e., a carbon-carbon sp triple bond. Unless otherwise specified, an alkynyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, ethynyl, propynyl, and the like.

The term “alkoxy” refers to an —O-alkyl radical. Thus, for example, alkoxy or alkoxyl can refer to groups of 1, 2, 3, 4, 5, 6, 7 or 8 carbon atoms of a straight, branched, cyclic configuration and combinations thereof attached to the parent structure through an oxygen atom. Examples include, but are not limited to, methoxy, ethoxy, propoxy, isopropoxy, cyclopropyloxy, cyclohexyloxy and the like. Lower-alkoxy refers to groups containing one to four carbons.

The term “cycloalkyl” as employed herein includes saturated monocyclic, bicyclic, tricyclic, or polycyclic hydrocarbon groups having 3 to 12 carbons, wherein any ring atom capable of substitution can be substituted by a substituent. Examples of cycloalkyl moieties include, but are not limited to, cyclopropyl, cyclobutyl, cyclopentyl, norbornyl, cyclohexyl, and adamantyl.

The term “carbocycle” as employed herein includes saturated, partially unsaturated or unsaturated monocyclic, bicyclic, tricyclic, or polycyclic hydrocarbon groups having 3 to 12 carbons, wherein any ring atom capable of substitution can be substituted by a substituent. Carbocycles can be aromatic, e.g., a phenyl group is an example of a carbocycle. A subset of carbocycles are non-aromatic carbocycles.

In some embodiments, two independent occurrences of a variable may be taken together with the atom(s) to which each variable is bound to form a 5-8-membered, heterocyclyl, aryl, or heteroaryl ring or a 3-8-membered cycloalkyl ring. Example rings that are formed when two independent occurrences of a substituent are taken together with the atom(s) to which each variable is bound include, but are not limited to the following: a) two independent occurrences of a substituent that are bound to the same atom and are taken together with that atom to form a ring, where both occurrences of the substituent are taken together with the atom to which they are bound to form a heterocyclyl, heteroaryl, carbocyclyl or aryl ring, wherein the group is attached to the rest of the molecule by a single point of attachment; and b) two independent occurrences of a substituent that are bound to different atoms and are taken together with both of those atoms to form a heterocyclyl, heteroaryl, carbocyclyl or aryl ring, wherein the ring that is formed has two points of attachment with the rest of the molecule. For example, where a phenyl group is substituted with two occurrences of ORₒ, as in Formula D1

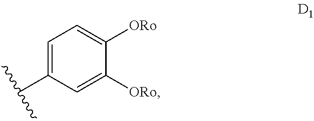

the two occurrences of ORₒ, where Rₒ, for example, is Me, are taken together with the carbon atoms to which they are bound to form a fused 6-membered oxygen containing ring as in Formula D2:

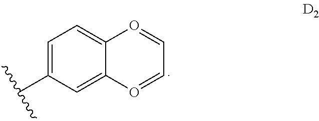

It will be appreciated that a variety of other rings can be formed when two independent occurrences of a substituent are taken together with the atom(s) to which each substituent is bound and that the examples detailed above are not intended to be limiting.

The term “substituents” refers to a group “substituted” on an alkyl, cycloalkyl, alkenyl, alkynyl, heterocyclyl, heterocycloalkenyl, cycloalkenyl, aryl, or heteroaryl group or other group at any atom of the group. The group can be singly or multiply substituted and where multiply substituted, the substituents are independent. Suitable substituents include, without limitation: F, Cl, Br, I, alkyl, alkenyl, alkynyl, alkoxy, acyloxy, halo, hydroxy, cyano, nitro, amino, SO₃H, sulfate, phosphate, perfluoroalkyl, perfluoroalkoxy, methylenedioxy, ethylenedioxy, carboxyl, oxo, thioxo, imino (alkyl, aryl, aralkyl), S(O)ₙ alkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, and combinations thereof), ester (alkyl, aralkyl, heteroaralkyl), amide (mono-, di-, alkyl, aralkyl, heteroaralkyl, and combinations thereof), sulfonamide (mono-, di-, alkyl, aralkyl, heteroaralkyl, and combinations thereof), unsubstituted aryl, unsubstituted heteroaryl, unsubstituted heterocyclyl, and unsubstituted cycloalkyl. In one aspect, the substituents on a group are independently any one single, or any subset of the aforementioned substituents. In some cases the substituents are selected from: F, Cl, Br and I. In other cases the substituents are selected from: halogen, optionally independently halogen substituted C₁-C₃ alkyl, optionally independently halogen substituted C₁-C₃ alkoxy, hydroxy, cyano, nitro and amino. In some cases the substituents are selected from aryl groups. In some cases the substituents are selected from heteroaryl groups. In some cases substituents are selected from: halogen, hydroxy, and C₁-C₃ alkyl. In some cases substituents are selected from: halogen, hydroxy, and C₁-C₃ alkyl and C₁-C₃ alkoxyl.

Unless only one of the isomers is drawn or named specifically, structures depicted herein are also meant to include all stereoisomeric (e.g., enantiomeric, diastereomeric, atropoisomeric and cis-trans isomeric) forms of the structure; for example, the R and S configurations for each asymmetric center, Ra and Sa configurations for each asymmetric axis, (Z) and (E) double bond configurations, and cis and trans conformational isomers. Therefore, single stereochemical isomers as well as racemates, and mixtures of enantiomers, diastereomers, and cis-trans isomers (double bond or conformational) of the present compounds are within the scope of the present disclosure. Unless otherwise stated, all tautomeric forms of the compounds of the present disclosure are within the scope of the disclosure.

The present disclosure also embraces the use of isotopically labeled compounds which are identical to those recited herein, but for the fact that one or more atoms are replaced by an atom having an atomic mass or mass number different from the atomic mass or mass number usually found in nature. All isotopes of any particular atom or element as specified are contemplated within the scope of the compounds of the invention, and their uses. Example isotopes that can be incorporated into compounds of the invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³F, ³⁵S, ¹⁸F, ³⁶CL, ¹²³I, and ¹²⁵I, respectively. Certain isotopically labeled compounds of the present invention (e.g., those labeled with ³H and ¹⁴C) are useful in compound and/or substrate tissue distribution assays. Tritiated (i.e., ³H) and carbon-14 (i.e., ¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron-emitting isotopes such as ¹⁵O, ¹³N, ¹¹C, and ¹⁸F are useful for positron emission tomography (PET) studies to examine substrate receptor occupancy. Isotopically labeled compounds of the present invention can generally be prepared by following procedures known to those having ordinary skill in the art, by substituting an isotopically labeled reagent for a non-isotopically labeled reagent.

The term “AADC” refers to an amino acid decarboxylase.

The term “COMT” refers to catechol-O-methyl transferase.

### Embodiments

In one aspect, the present invention provides a method of treating or preventing a sleep disorder, impulse control disorder (ICD) and/or dopamine dysregulation syndrome (DDS) arising from the treatment of a patient suffering from Parkinson's Disease (PD), restless legs syndrome (RLS) or periodic limb movement disorder (PLMD) with a dopaminergic agent, comprising administering a therapeutically effective amount of a FAAH inhibitor to said patient.

In another aspect, the present invention provides a method of treating Parkinson's Disease (PD), restless legs syndrome (RLS) or periodic limb movement disorder (PLMD) while preventing or ameliorating an impulse control disorder (ICD) and/or dopamine dysregulation syndrome (DDS) in a patient in need thereof, comprising administering a therapeutically effective amount of a dopaminergic agent and a therapeutically effective amount of a FAAH inhibitor to said patient.

In another aspect, the present invention provides a pharmaceutical composition comprising a dopaminergic agent and a FAAH inhibitor.

In another aspect, the present invention provides a pharmaceutical composition comprising a FAAH inhibitor, optionally in combination with one or more other drugs, for use in the treatment of PD, RLS or PLMD.

In another aspect, the present invention provides a kit comprising at least two separate unit dosage forms (A) and (B), wherein (A) is a dopaminergic agent, a pharmaceutically acceptable salt thereof, or a pharmaceutical composition thereof, and (B) is a FAAH inhibitor, a pharmaceutically acceptable salt thereof, or a pharmaceutical composition thereof.

In another aspect, the present invention provides for the use of a FAAH inhibitor or a pharmaceutically acceptable salt thereof for the manufacture of a medicament for the treatment or prevention of a sleep disorder, an impulse control disorder (ICD) and/or dopamine dysregulation syndrome (DDS) arising from the treatment of a patient suffering from Parkinson's Disease (PD), restless legs syndrome (RLS), or periodic limb movement disorder (PLMD) with a dopaminergic agent.

In some embodiments, the methods or uses treat or prevent DDS. In a further embodiment, DDS is characterized by punding, stereotypies, hobbyism and/or walkabouts.

In some embodiments, the methods or uses treat or prevent an ICD. In a further embodiment, the ICD is selected from pathological gambling, an eating disorder, compulsive shopping and/or hypersexuality.

In some embodiments, the methods or uses treat or prevent a sleep disorder arising from the treatment of a patient suffering from Parkinson's Disease (PD), restless legs syndrome (RLS), or periodic limb movement disorder (PLMD) with a dopaminergic agent. In a further embodiment, the sleep disorder is characterized by impaired sleeping, including restlessness, insomnia, vivid dreaming and nightmares.

In other embodiments, the methods or uses treat or prevent one or more of a DDS, an ICD, or a sleep disorder.

In some embodiments of the above methods and uses, the patient is suffering from PD. In some embodiments of the above methods and uses, the patient is suffering from RLS. In some embodiments of the above methods and uses, the patient is suffering from PLMD. In some embodiments, the patient is suffering from RLS and PLMD. In some embodiments, the patient is a human.

In some embodiments of the above methods, pharmaceutical compositions, kits and uses, the dopaminergic agent is a dopamine replacement agent, a dopamine agonist, a dopamine uptake inhibitor or a monoamine oxidase inhibitor. In some embodiments, the dopaminergic agent is a dopamine replacement agent. In a further embodiment, the dopamine replacement agent comprises melevodopa or L-3,4-dihydroxyphenylalanine (levodopa, L-DOPA). In yet a further embodiment, the dopamine replacement agent further comprises an AADC enzyme inhibitor. In still a further embodiment, the AADC enzyme inhibitor is carbidopa or benserazide.

In further embodiments of the above methods, pharmaceutical compositions, kits and uses, the dopamine replacement agent is levodopa combined with an AADC enzyme inhibitor such as carbidopa, benserazide (e.g., levodopa/carbidopa (Sinemet® or Atamet®), levodopa/benserazide (Madopar®), extended release levodopa/carbidopa (Sinemet CR®), or extended release levodopa/benserazide (Madopar HBS®). In a further embodiment, the dopamine replacement agent is levodopa combined with an AADC and a COMT enzyme inhibitor (e.g. levodopa/carbidopa/entacapone (Stalevo®)).

In other embodiments, the dopaminergic agent is a dopamine agonist. In further embodiments, the dopamine agonist is bromocriptine (Parlodel®), pergolide (Permax®), pramipexole (Mirapex®), ropinirole (Requip®), rotigotine (Neupro®), cabergoline (Dostinex®), apomorphine (Apokyn®), lisuride (Dopergine®) or talipexole.

In other embodiments, the dopaminergic agent is a dopamine uptake inhibitor. In further embodiments, the dopamine uptake inhibitor is BLS-602/BLS-605 or SEP-226330, bupropion (Wellbutrin®, Zyban®), dexmethylphenidate (Focalin®) or methylphenidate (Ritalin®, Concerta®).

In other embodiments, the dopaminergic agent is a monoamine oxidase inhibitor. In further embodiments, the monoamine oxidase inhibitor is rasagiline, selegiline or safinamide.

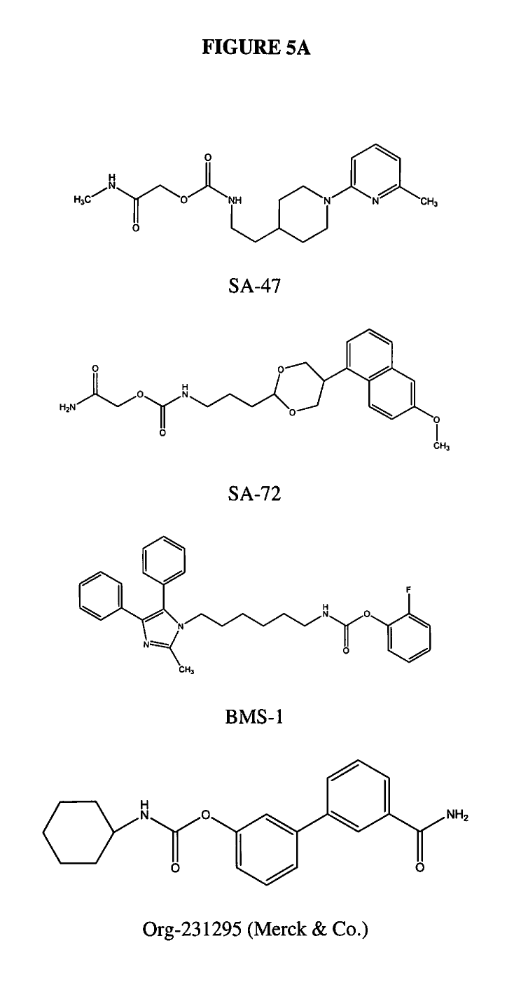

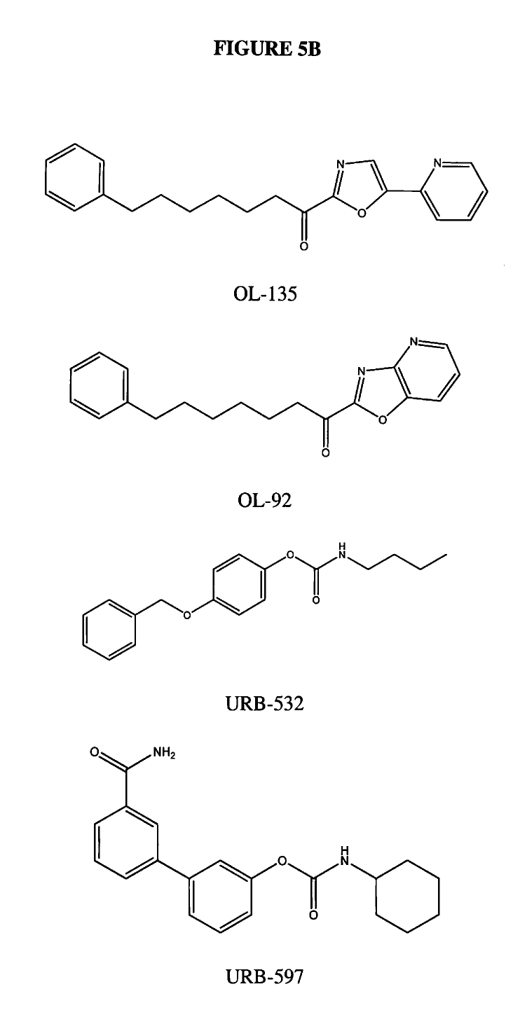

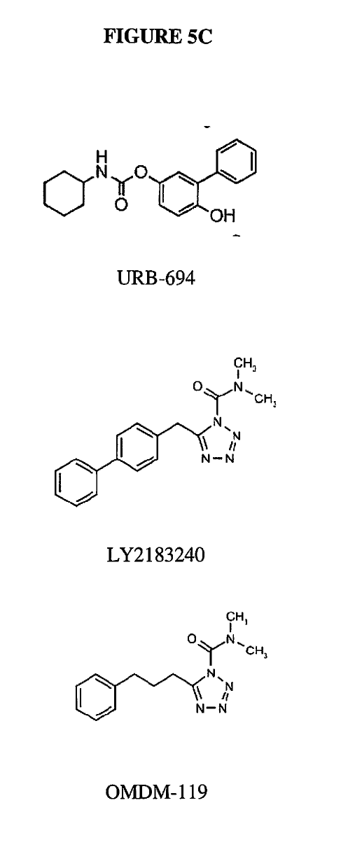

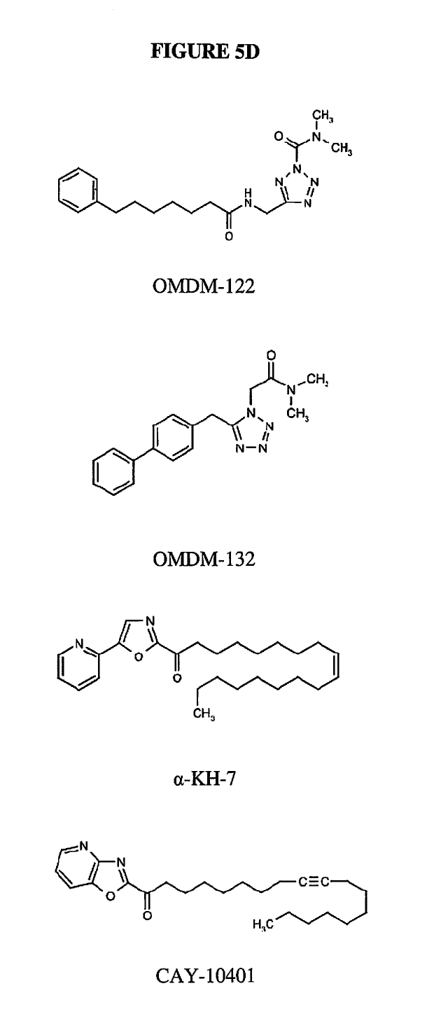

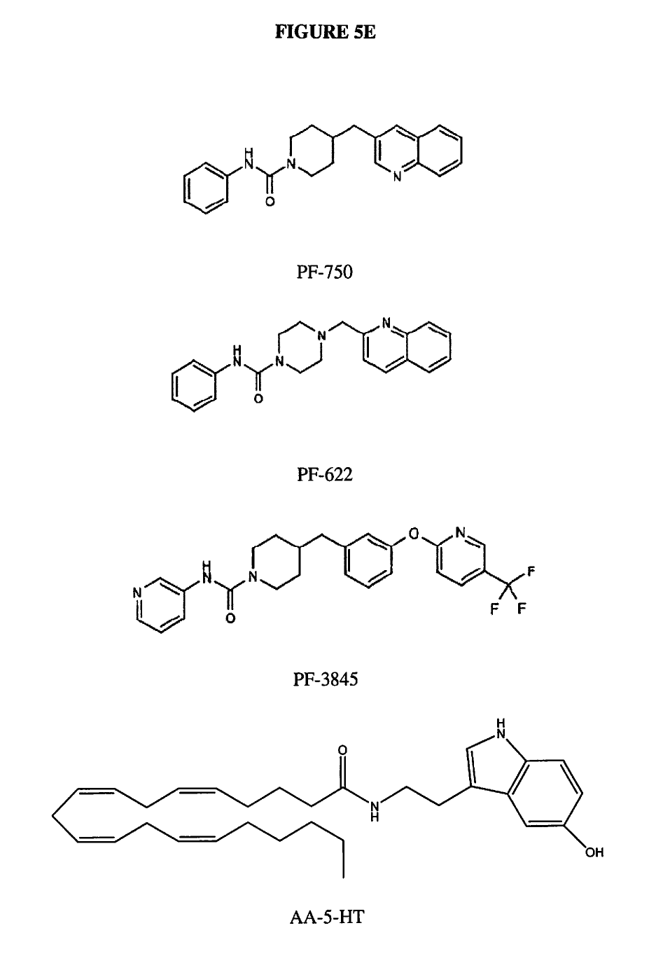

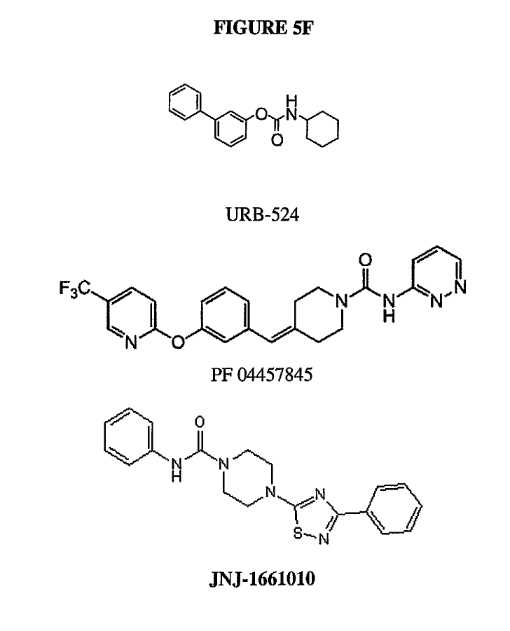

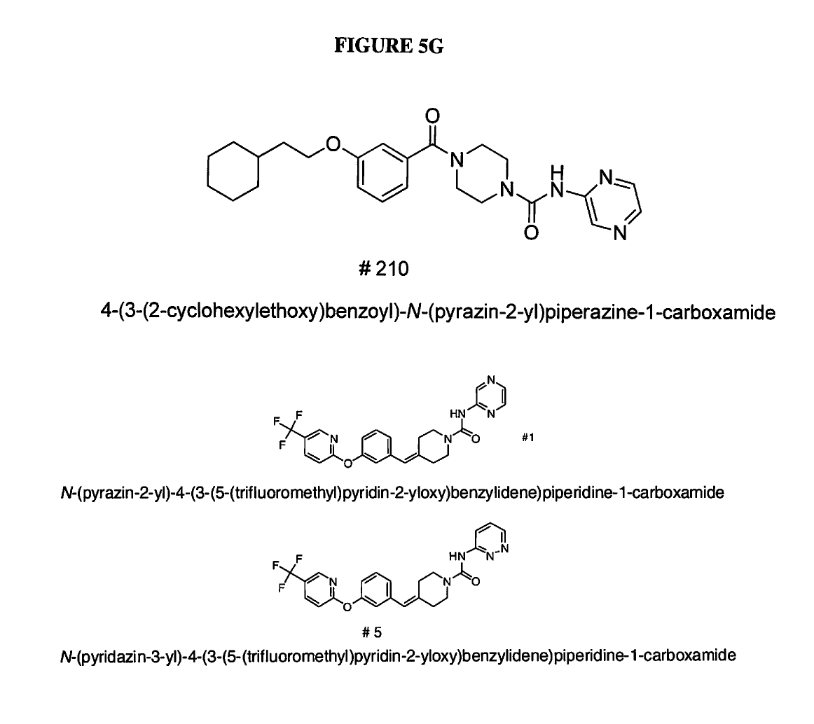

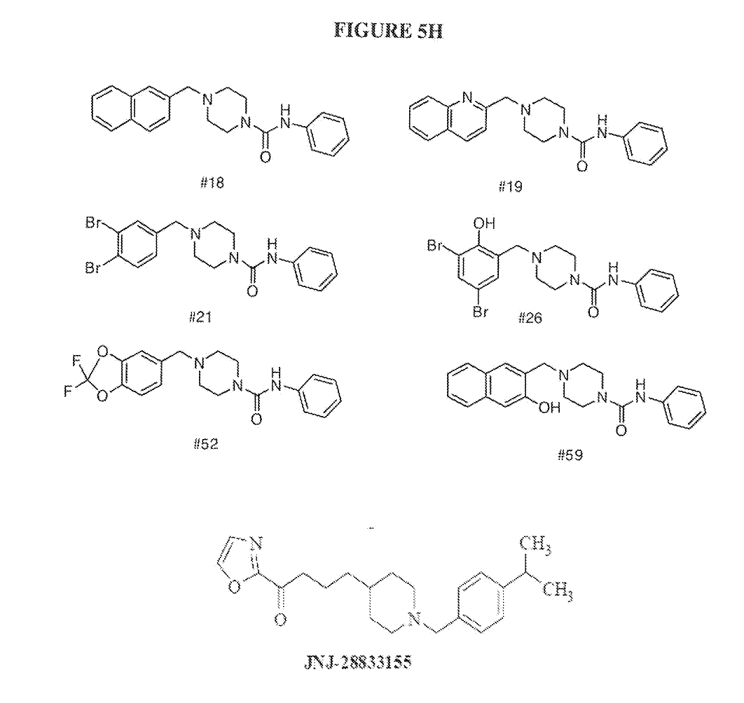

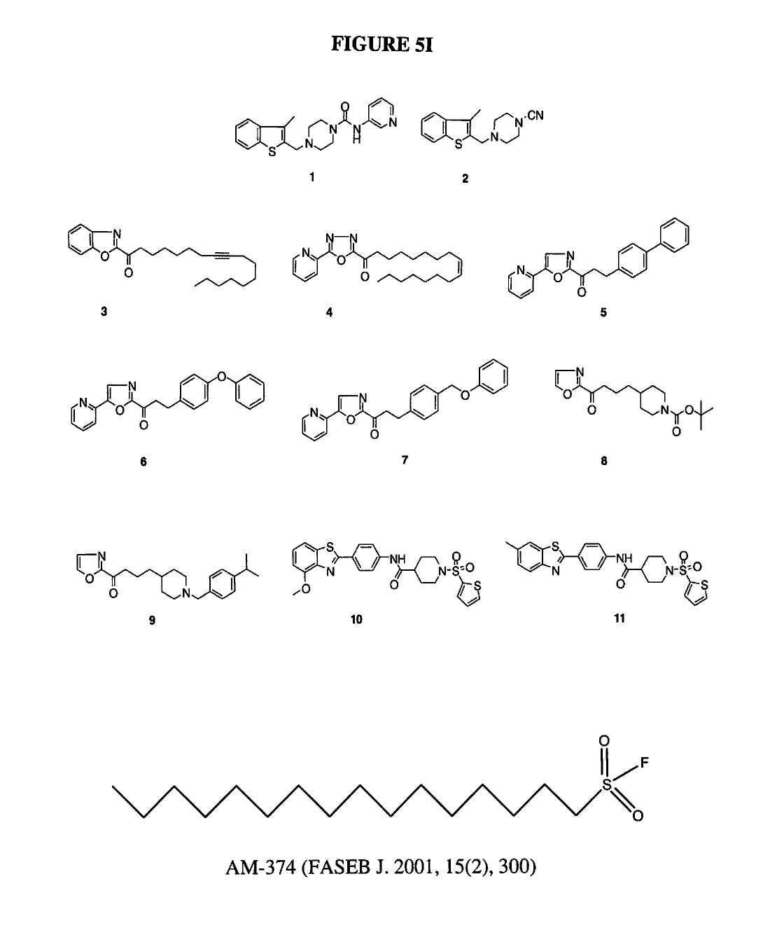

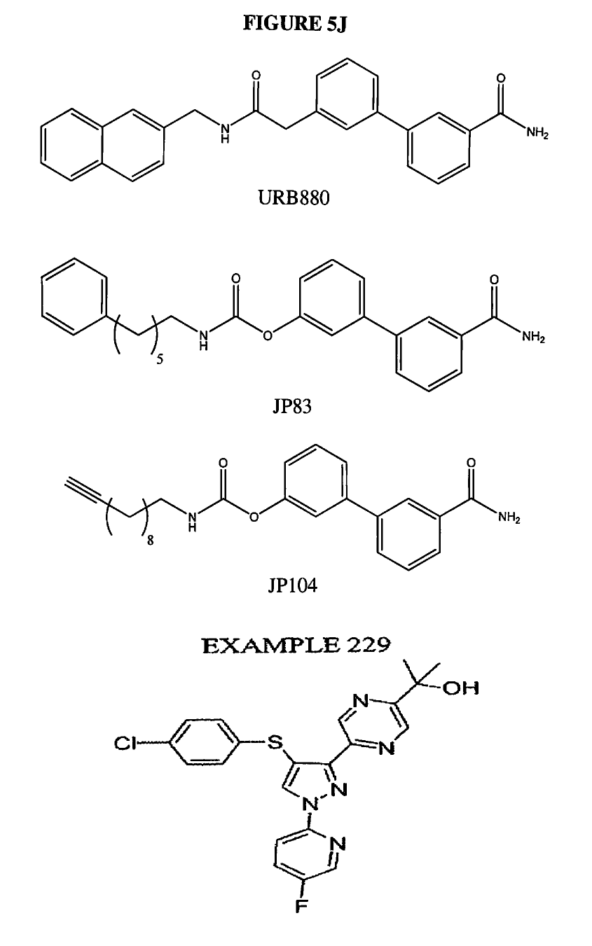

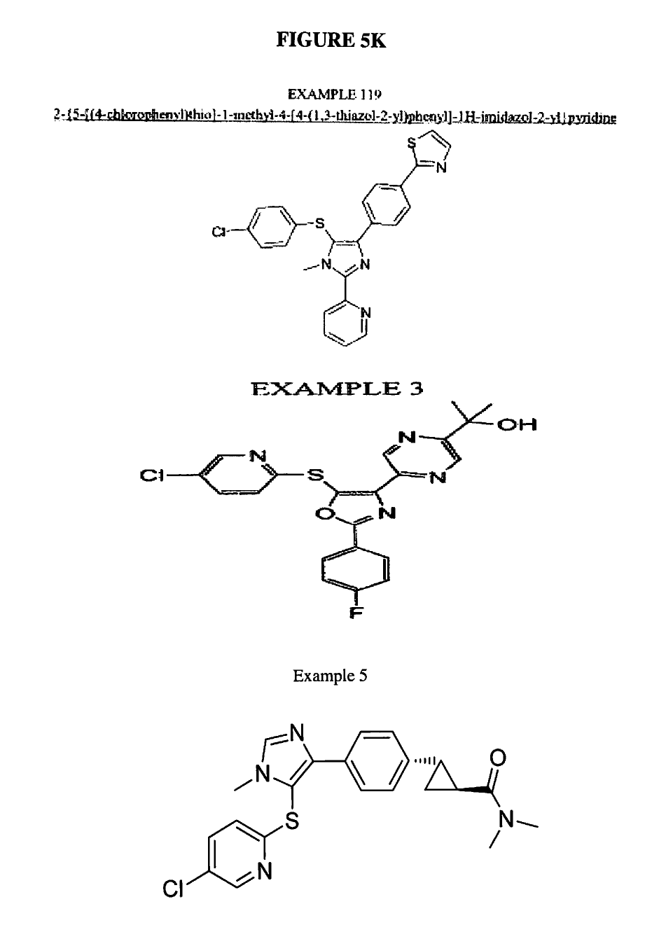

In some embodiments of the above methods, pharmaceutical compositions, kits and uses, the FAAH inhibitor is selected from those provided in FIGS. 5A-5K. In some embodiments of the above methods, pharmaceutical compositions, kits and uses, the FAAH inhibitor is SA-47, SA-72, BMS-1, Org-23295, OL-135, OL-92, URB-597, URB-532, URB-694, URB-524, LY2183240, OL-135, OMDM-119, OMDM-122, OMDM-132, α-KH-7, AA-5-HT, CAY-10401, PF-750, PF-3845, PF-622, BMS-469908, SSR-411298, TK-25, PF-04457845, JNJ-245, JNJ-28833155, JNJ-1661010, compound 210 from EP 2065369, compounds 1, 4 or 5 from WO2008/047229, compounds 18, 19, 21, 26, 52 or 59 from WO 2006/074025, compound 229 from WO 2009/151991, compound 129 from WO 2009/152025, compound 3 from WO2010/017079, example #5 from WO2010/101274 or compounds 1-11 from S. Pillarisetti et al., “Pain and beyond: fatty acid amides and fatty acid amide hydrolase inhibitors in cardiovascular and metabolic diseases”, Drug Discov. Today (2009), doi:10.1016/j.drudis.2009.08.002.

In some embodiments of the above methods, pharmaceutical compositions, kits and uses, the FAAH inhibitor is a compound disclosed in WO2010/141817, WO2010/141809, WO2010/135360, WO2010/130945, WO2010/130944, WO2010/130943, WO2010/124113, WO2010/117014, WO2010/118159, WO2010/118155, WO2010/089510, WO2010/074588, WO2010/074587, WO2010/0068453, WO2010/0068452, WO2010/064597, WO2010/058318, WO2010/059610, WO2010/055267, WO2010/053120, WO2010/049841, WO2010/039186, WO2010/017079, WO2010/010288, WO2010/007966, WO2010/005572, WO2010/101274, WO2009/154785, WO2009/109504, WO2009/084970, WO 2009/151991, WO 2009/152025, WO 2009/127943, WO 2009/127944, WO 2009/127946, WO 2009/127949, WO 2009/127948, WO 2009/126691, WO 2009/109743, WO 2009/105220, US 2009/0163508, EP 2065369, WO2008/157740, US 2009/0118503, US 2009/0111778, WO 2009/051666, US 2009/0030074, WO 2009/011904, WO2008/150492, WO2008/145839, WO2008/147553, US2010/41651, WO2008/745843, US2010/41670, WO2008/129129, US2009/099240, WO 2008/047229, WO 2008/153752, US 2008/0312226, WO2008/020866, WO 2008/022976, WO 2008/100977, WO2008/030752, WO2008/042892, WO2008/030532, US 2008/0045513, WO2008/021625, US2008/089845, US2008/119549, WO2007/098142, WO2007/020888, WO2007/070892, US2009/48263, WO2006/117461, US2008/103197, WO2006/044617, WO 2006/054652, WO 2006/074025, WO2006/117461, US2007/027141, US 2006/0173184, WO 2003/065989, WO 2004/033422, WO2004/033652, WO2004/053066, WO2004/099176, US2006/89344, WO 2006/088075, EP1923388, WO 2008/063300, WO 2005/090322, US 2009/0143365, WO 2007/140005, WO2007/005510, US2007/0004741, WO 2006/0258700 or WO 2007/061862.

In some embodiments of the above methods, pharmaceutical compositions, kits and uses, the FAAH inhibitor is a compound of formula I:

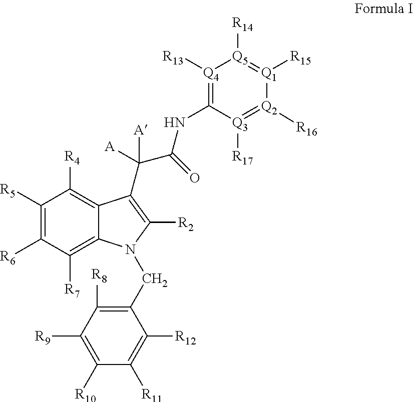

wherein:
each of Q₁, Q₂, Q₃, Q₄, and Q₅ are independently N or C;
A and A′ are independently: hydroxyl or an optionally independently substituted C₁ to C₃ alkoxy or A and A′ taken together are ═O, ═N(OH) or ═NOCH₃ or A and A′ together with the carbon to which they are attached form a cyclic ketal containing a total of 4 or 5 carbon atoms which can be optionally independently substituted;
R₂ is halogen, hydroxyl, —NO₂, an optionally independently substituted C₁-C₅ alkyl, an optionally independently substituted C₁-C₅ alkoxy, an optionally independently substituted C₂-C₅ alkenyl, an optionally independently substituted C₂-C₅ alkynyl, —CN, —C(O)OH, an optionally independently substituted cyclopropyl, —C(O)NR₂ₐR_{2b}, or —NR₂ₐR_{2b}, wherein R₂ₐ and R_{2b} are independently H or C₁-C₃ alkyl;
each of R₄, R₅, R₆ and R₇ is independently: H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b}, or —NRₐR_{b}, wherein Rₐ and R_{b} are independently H, an optionally independently substituted C₁-C₆ alkyl, or an optionally independently substituted C₃-C₆ cycloalkyl;
each of R₈, R₉, R₁₀, R₁₁ and R₁₂ is independently: H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b}, or —NRₐR_{b}, wherein Rₐ and R_{b} are independently H, an optionally independently substituted C₁-C₆ alkyl, or an optionally independently substituted C₃-C₆ cycloalkyl;
when Q₅ is C, R₁₄ is selected from H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b}, or —NRₐR_{b}, wherein Rₐ and R_{b} are independently H, an optionally independently substituted C₁-C₆ alkyl, or an optionally independently substituted C₃-C₆ cycloalkyl;
when Q₅ is N, R₁₄ is missing;
when Q₂ is C, R₁₆ is selected from H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b}, or —NRₐR_{b}, wherein Rₐ and R_{b} are independently H, an optionally independently substituted C₁-C₆ alkyl, or an optionally independently substituted C₃-C₆ cycloalkyl;
when Q₂ is N, R₁₆ is missing;
when Q₁ is C, R₁₅ is selected from H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b}, or —NRₐR_{b}, wherein Rₐ and R_{b} are independently H, an optionally independently substituted C₁-C₆ alkyl, or an optionally independently substituted C₃-C₆ cycloalkyl;
when Q₁ is N, R₁₅ is missing;
when Q₄ is C, R₁₃ is selected from H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b}, or —NRₐR_{b}, wherein Rₐ and R_{b} are independently H, an optionally independently substituted C₁-C₆ alkyl, or an optionally independently substituted C₃-C₆ cycloalkyl;
when Q₄ is N, R₁₃ is missing;
when Q₃ is C, R₁₇ is selected from H, a halogen, —NO₂, —CN, —C(O)OH, hydroxyl, an optionally independently substituted C₁-C₅ alkyl, an optionally independently substituted C₂-C₅ alkenyl, an optionally independently substituted C₂-C₅ alkynyl, an optionally independently substituted C₁-C₅ alkoxy, —C(O)NRₐR_{b}, or —NRₐR_{b}, wherein Rₐ and R_{b} are independently H, optionally independently substituted C₁-C₆ alkyl, or an optionally independently substituted C₃-C₆ cycloalkyl;
and
when Q₃ is N, R₁₇ is missing.

In some embodiments, when Q₁, Q₂, Q₃, Q₄, and Q₅ are C; R₂ is methyl; and A and A′ taken together are ═O, then

(1) R₁₅ is not C(O)NH₂ and R₁₀ is not Cl; (2) R₈, R₉, R₁₀, R₁₁, and R₁₂ are not all H and R₁₃ and R₁₇ are not both methyl; and (3) R₈, R₉, R₁₀, R₁₁, R₁₂, R₁₃, R₁₄, R₁₅, R₁₆, R₁₇ are not all H.

In further embodiments, the FAAH inhibitor is a compound of Formula A-2, Formula A-3 or Formula A-4:

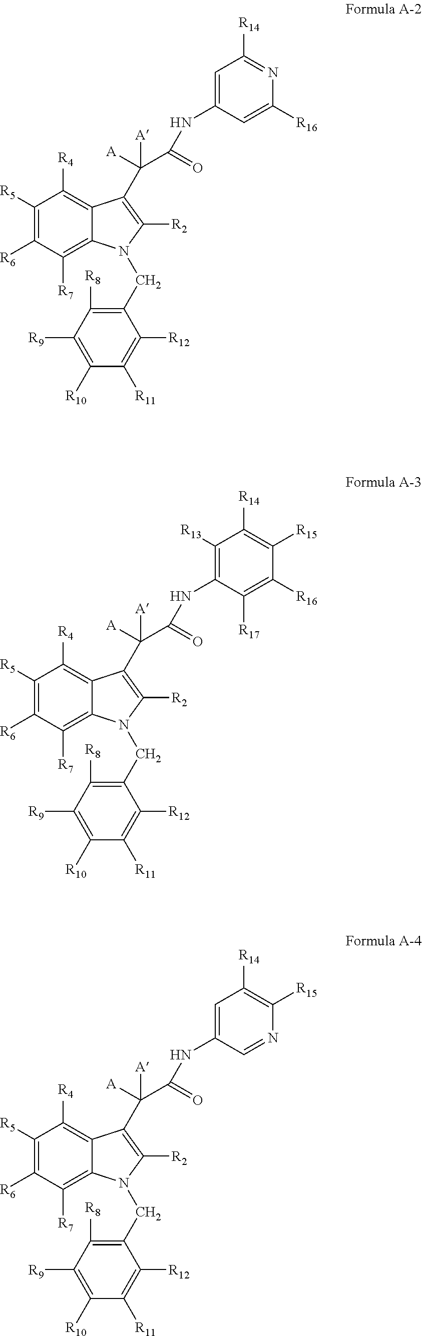

In further embodiments, the FAAH inhibitor is a compound of Formula A-5 or Formula A-7:

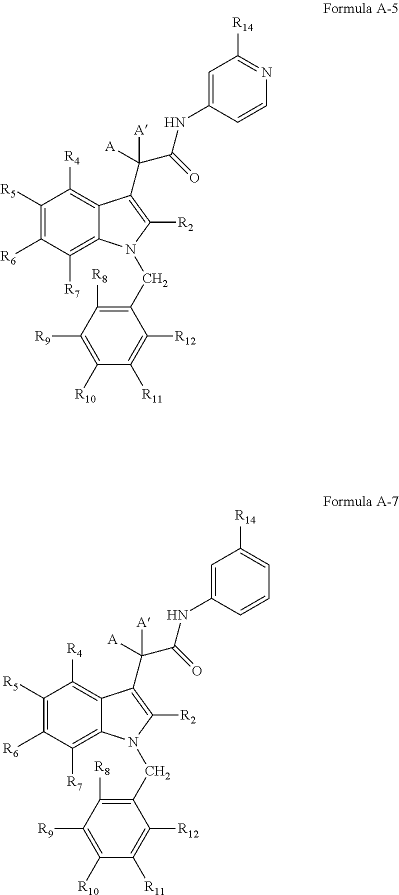

In yet further embodiments, A and A′ taken together are ═O in said compound.

In yet further embodiments, R₂ is an optionally independently halogen substituted C₁-C₃ alkyl or cyclopropyl in said compound. In still further embodiments, R₂ is methyl in said compound.

In yet further embodiments, one or two of R₈, R₉, R₁₀, R₁₁ and R₁₂ are halogen and the rest are H in said compound. In still further embodiments, R₁₀ is Cl or F and R₈, R₉, R₁₁ and R₁₂ are H.

In yet further embodiments, R₄ and R₇ are H in said compound.

In yet further embodiments, R₆ is H in said compound.

In yet further embodiments, R₅ is selected from: ethoxy, methoxy, ethyl, methyl, halogen and H in said compound. In still further embodiments, R₅ is methoxy or methyl.

In yet further embodiments, each of R₁₃, R₁₅, R₁₆ and R₁₇ is independently selected from H, a halogen, —NO₂, —CN, —C(O)OH, hydroxyl, a C₁-C₅ alkyl, a C₂-C₅ alkenyl, a C₂-C₅ alkynyl, a C₁-C₅ alkoxy, —C(O)NRₐR_{b}, or —NRₐR_{b}, wherein Rₐ and R_{b} are independently H, a C₁-C₆ alkyl, or a C₃-C₆ cycloalkyl in said compound.

In yet further embodiments, R₁₄ is halogen or an optionally independently substituted methoxy and both R₁₃ and R₁₇ are H in said compound. In still further embodiments, R₁₄ is halogen or an optionally independently substituted methoxy in said compound. In still further embodiments, R₁₄ is Cl, F or —OCH₃.

In some embodiments of the above methods, pharmaceutical compositions, kits and uses, the FAAH inhibitor is selected from the following:
2-[1-(4-chlorobenzyl)-2-methyl-1H-indol-3-yl]-N-(2-chloropyridin-4-yl)-2-oxoacetamide
2-[1-(4-chlorobenzyl)-2-methyl-1H-indol-3-yl]-N-(3-methoxyphenyl)-2-oxoacetamide
2-[1-(4-chlorobenzyl)-5-methoxy-2-methyl-1H-indol-3-yl]-2-oxo-N-pyridin-2-ylacetamide
2-[2-chloro-1-(4-chlorobenzyl)-5-methoxy-1H-indol-3-yl]-2-oxo-N-pyridin-3-ylacetamide
2-[1-(4-chlorobenzyl)-5-methoxy-2-methyl-1H-indol-3-yl]-2-oxo-N-pyridin-4-ylacetamide
2-[1-(4-chlorobenzyl)-2,5-dimethyl-1H-indol-3-yl]-2-oxo-N-pyridin-4-ylacetamide
2-[1-(4-chlorobenzyl)-5-methoxy-2-methyl-1H-indol-3-yl]-2-oxo-N-phenylacetamide
2-[2-chloro-1-(4-chlorobenzyl)-5-methoxy-1H-indol-3-yl]-2-oxo-N-pyridin-4-ylacetamide
2-[2-chloro-1-(4-chlorobenzyl)-5-methoxy-1H-indol-3-yl]-2-oxo-N-pyrimidin-4-ylacetamide
2-[2-chloro-1-(4-chlorobenzyl)-5-methoxy-1H-indol-3-yl]-N-(2-chloropyridin-4-yl)-2-oxoacetamide
2-[1-(4-chlorobenzyl)-5-methoxy-2-methyl-1H-indol-3-yl]-N-(2-chloropyridin-4-yl)-2-oxoacetamide
2-[1-(4-chlorobenzyl)-5-ethoxy-2-methyl-1H-indol-3-yl]-2-oxo-N-phenylacetamide
2-[1-(4-chlorobenzyl)-5-methoxy-2-methyl-1H-indol-3-yl]-N-(3-methoxyphenyl)-2-oxoacetamide
2-[1-(4-chlorobenzyl)-2,5-dimethyl-1H-indol-3-yl]-2-oxo-N-phenylacetamide
2-[1-(2,4-dichlorobenzyl)-5-methoxy-2-methyl-1H-indol-3-yl]-2-oxo-N-pyridin-4-ylacetamide
2-[1-(4-chlorobenzyl)-5-methoxy-2-methyl-1H-indol-3-yl]-N-(3-chlorophenyl)-2-oxoacetamide
2-[1-(4-chlorobenzyl)-5-methoxy-2-methyl-1H-indol-3-yl]-2-oxo-N-pyrimidin-4-ylacetamide
2-[1-(4-chlorobenzyl)-2,5-dimethyl-1H-indol-3-yl]-2-oxo-N-pyridin-3-ylacetamide
2-[1-(4-chlorobenzyl)-5-methoxy-2-methyl-1H-indol-3-yl]-2-oxo-N-pyridin-3-ylacetamide
2-[1-(2,4-dichlorobenzyl)-5-methoxy-2-methyl-1H-indol-3-yl]-2-oxo-N-pyridin-3-ylacetamide
2-[1-(4-chlorobenzyl)-5-methoxy-2-methyl-1H-indol-3-yl]-N-(4-chlorophenyl)-2-oxoacetamide
2-[1-(4-chlorobenzyl)-5-methoxy-2-methyl-1H-indol-3-yl]-N-(4-methoxyphenyl)-2-oxoacetamide
2-[5-chloro-1-(4-chlorobenzyl)-2-methyl-1H-indol-3-yl]-2-oxo-N-pyridin-2-ylacetamide
2-[1-(4-chlorobenzyl)-2-isopropyl-5-methoxy-1H-indol-3-yl]-2-oxo-N-pyridin-4-ylacetamide
2-[1-(4-chlorobenzyl)-5-methoxy-2-methyl-1H-indol-3-yl]-N-(2-chlorophenyl)-2-oxoacetamide
2-[1-(4-chlorobenzyl)-2-isopropyl-5-methoxy-1H-indol-3-yl]-2-oxo-N-pyridin-3-ylacetamide
2-[1-(4-chlorobenzyl)-2-isopropyl-5-methoxy-1H-indol-3-yl]-2-oxo-N-phenylacetamide
2-[1-(4-chlorobenzyl)-5-methoxy-2-methyl-1H-indol-3-yl]-N-(2-methoxyphenyl)-2-oxoacetamide
2-[1-(4-chlorobenzyl)-5-ethoxy-2-methyl-1H-indol-3-yl]-2-oxo-N-pyridin-4-ylacetamide
2-[1-(4-chlorobenzyl)-2-methyl-1H-indol-3-yl]-2-oxo-N-pyridin-4-ylacetamide
2-[1-(4-chlorobenzyl)-5-hydroxy-2-methyl-1H-indol-3-yl]-2-oxo-N-phenylacetamide
2-[1-(4-chlorobenzyl)-2-methyl-1H-indol-3-yl]-2-oxo-N-pyridin-3-ylacetamide
2-[1-(4-chlorobenzyl)-2-methyl-1H-indol-3-yl]-2-oxo-N-phenylacetamide
N-(3-chlorophenyl)-2-[1-(2,4-dichlorobenzyl)-5-methoxy-2-methyl-1H-indol-3-yl]-2-oxoacetamide
2-[1-(2,4-dichlorobenzyl)-5-methoxy-2-methyl-1H-indol-3-yl]-N-(3-methoxyphenyl)-2-oxoacetamide
2-[1-(2,4-dichlorobenzyl)-5-methoxy-2-methyl-1H-indol-3-yl]-N-(5-methoxy-2-methylphenyl)-2-oxoacetamide
2-[1-(2,4-dichlorobenzyl)-5-methoxy-2-methyl-1H-indol-3-yl]-2-oxo-N-pyrimidin-4-ylacetamide
2-[1-(2,4-dichlorobenzyl)-5-methoxy-2-methyl-1H-indol-3-yl]-2-oxo-N-phenylacetamide
2-[1-(4-chlorobenzyl)-5-methoxy-2-methyl-1H-indol-3-yl]-N-(3-hydroxypyridin-2-yl)-2-oxoacetamide
2-[1-(4-chlorobenzyl)-2-methyl-1H-indol-3-yl]-2-oxo-N-pyrimidin-4-ylacetamide
2-[1-(4-chlorobenzyl)-2-methyl-1H-indol-3-yl]-N-(3-chlorophenyl)-2-oxoacetamide
2-[1-(4-chlorobenzyl)-5-hydroxy-2-methyl-1H-indol-3-yl]-2-oxo-N-pyridin-4-ylacetamide
2-[1-(4-chlorobenzyl)-5-methoxy-2-methyl-1H-indol-3-yl]-N-(2-fluorophenyl)-2-oxoacetamide
2-[1-(4-chlorobenzyl)-5-methoxy-2-methyl-1H-indol-3-yl]-N-(3,5-dichlorophenyl)-2-oxoacetamide
2-[1-(4-chlorobenzyl)-5-methoxy-2-methyl-1H-indol-3-yl]-N-(3-fluorophenyl)-2-oxoacetamide
2-[1-(4-chlorobenzyl)-5-methoxy-2-methyl-1H-indol-3-yl]-N-(4-fluorophenyl)-2-oxoacetamide
2-[1-(4-chlorobenzyl)-5-methoxy-2-methyl-1H-indol-3-yl]-N-(6-methoxypyrimidin-4-yl)-2-oxoacetamide
2-[2-chloro-1-(4-chlorobenzyl)-5-methoxy-1H-indol-3-yl]-N-(3-chlorophenyl)-2-oxoacetamide
2-[2-chloro-1-(4-chlorobenzyl)-5-methoxy-1H-indol-3-yl]-N-(3-methoxyphenyl)-2-oxoacetamide
2-[5-chloro-1-(4-chlorobenzyl)-2-methyl-1H-indol-3-yl]-2-oxo-N-pyridin-4-ylacetamide
2-[5-chloro-1-(4-chlorobenzyl)-2-methyl-1H-indol-3-yl]-2-oxo-N-pyrimidin-4-ylacetamide
2-[5-chloro-1-(4-chlorobenzyl)-2-methyl-1H-indol-3-yl]-N-(2-chloropyridin-4-yl)-2-oxoacetamide
2-[5-chloro-1-(4-chlorobenzyl)-2-methyl-1H-indol-3-yl]-N-(3-chlorophenyl)-2-oxoacetamide
2-[5-chloro-1-(4-chlorobenzyl)-2-methyl-1H-indol-3-yl]-N-(3-methoxyphenyl)-2-oxoacetamide
2-(1-benzyl-2,5-dimethyl-1H-indol-3-yl)-N-(2-methoxypyridin-4-yl)-2-oxoacetamide
2-(1-benzyl-2-methyl-1H-indol-3-yl)-N-(2-methoxypyridin-4-yl)-2-oxoacetamide
2-(1-benzyl-5-methoxy-2-methyl-1H-indol-3-yl)-N-(2-methoxypyridin-4-yl)-2-oxoacetamide
2-[1-(2,4-dichlorobenzyl)-2,5-dimethyl-1H-indol-3-yl]-N-(2-methoxypyridin-4-yl)-2-oxoacetamide
2-[1-(2,4-dichlorobenzyl)-2,5-dimethyl-1H-indol-3-yl]-N-(3-fluorophenyl)-2-oxoacetamide
2-[1-(2,4-dichlorobenzyl)-2-methyl-1H-indol-3-yl]-N-(2-methoxypyridin-4-yl)-2-oxoacetamide
2-[1-(2,4-dichlorobenzyl)-5-fluoro-2-methyl-1H-indol-3-yl]-N-(2-methoxypyridin-4-yl)-2-oxoacetamide
2-[1-(2,4-dichlorobenzyl)-5-methoxy-2-methyl-1H-indol-3-yl]-N-(2-methoxypyridin-4-yl)-2-oxoacetamide
2-[1-(2,4-difluorobenzyl)-2,5-dimethyl-1H-indol-3-yl]-N-(2-methoxypyridin-4-yl)-2-oxoacetamide
2-[1-(2,4-difluorobenzyl)-2-methyl-1H-indol-3-yl]-N-(2-methoxypyridin-4-yl)-2-oxoacetamide
2-[1-(2,4-difluorobenzyl)-5-methoxy-2-methyl-1H-indol-3-yl]-N-(2-methoxypyridin-4-yl)-2-oxoacetamide
2-[1-(2-chloro-4-fluorobenzyl)-2,5-dimethyl-1H-indol-3-yl]-N-(2-methoxypyridin-4-yl)-2-oxoacetamide
2-[1-(2-chloro-4-fluorobenzyl)-2-methyl-1H-indol-3-yl]-N-(2-methoxypyridin-4-yl)-2-oxoacetamide
2-[1-(2-chloro-4-fluorobenzyl)-5-methoxy-2-methyl-1H-indol-3-yl]-N-(2-methoxypyridin-4-yl)-2-oxoacetamide
2-[1-(2-chlorobenzyl)-5-methoxy-2-methyl-1H-indol-3-yl]-N-(2-methoxypyridin-4-yl)-2-oxoacetamide
2-[1-(3-chlorobenzyl)-5-methoxy-2-methyl-1H-indol-3-yl]-N-(2-methoxypyridin-4-yl)-2-oxoacetamide
2-[1-(4-chloro-2-fluorobenzyl)-2,5-dimethyl-1H-indol-3-yl]-N-(2-methoxypyridin-4-yl)-2-oxoacetamide
2-[1-(4-chloro-2-fluorobenzyl)-2-methyl-1H-indol-3-yl]-N-(2-methoxypyridin-4-yl)-2-oxoacetamide
2-[1-(4-chloro-2-fluorobenzyl)-5-methoxy-2-methyl-1H-indol-3-yl]-N-(2-methoxypyridin-4-yl)-2-oxoacetamide
2-[1-(4-chlorobenzyl)-2,5-dimethyl-1H-indol-3-yl]-2-oxo-N-pyrimidin-4-ylacetamide
2-[1-(4-chlorobenzyl)-2,5-dimethyl-1H-indol-3-yl]-N-(2-chloropyridin-4-yl)-2-oxoacetamide
2-[1-(4-chlorobenzyl)-2,5-dimethyl-1H-indol-3-yl]-N-(2-methoxypyridin-4-yl)-2-oxoacetamide
2-[1-(4-chlorobenzyl)-2,5-dimethyl-1H-indol-3-yl]-N-(3-chlorophenyl)-2-oxoacetamide
2-[1-(4-chlorobenzyl)-2,5-dimethyl-1H-indol-3-yl]-N-(3-methoxyphenyl)-2-oxoacetamide
2-[1-(4-chlorobenzyl)-2-methyl-1H-indol-3-yl]-N-(2-methoxypyridin-4-yl)-2-oxoacetamide
2-[1-(4-chlorobenzyl)-5-ethoxy-2-methyl-1H-indol-3-yl]-N-(2-chloropyridin-4-yl)-2-oxoacetamide
2-[1-(4-chlorobenzyl)-5-ethoxy-2-methyl-1H-indol-3-yl]-N-(2-methoxypyridin-4-yl)-2-oxoacetamide
2-[1-(4-chlorobenzyl)-5-ethoxy-2-methyl-1H-indol-3-yl]-N-(3-methoxyphenyl)-2-oxoacetamide
2-[1-(4-chlorobenzyl)-5-fluoro-2-methyl-1H-indol-3-yl]-2-oxo-N-pyridin-4-ylacetamide
2-[1-(4-chlorobenzyl)-5-fluoro-2-methyl-1H-indol-3-yl]-2-oxo-N-pyrimidin-4-ylacetamide
2-[1-(4-chlorobenzyl)-5-fluoro-2-methyl-1H-indol-3-yl]-N-(2-chloropyridin-4-yl)-2-oxoacetamide
2-[1-(4-chlorobenzyl)-5-fluoro-2-methyl-1H-indol-3-yl]-N-(2-methoxypyridin-4-yl)-2-oxoacetamide
2-[1-(4-chlorobenzyl)-5-fluoro-2-methyl-1H-indol-3-yl]-N-(3-chlorophenyl)-2-oxoacetamide
2-[1-(4-chlorobenzyl)-5-fluoro-2-methyl-1H-indol-3-yl]-N-(3-methoxyphenyl)-2-oxoacetamide
2-[1-(4-chlorobenzyl)-5-methoxy-2-methyl-1H-indol-3-yl]-2-oxo-N-[3-(trifluoromethoxy)phenyl]acetamide
2-[1-(4-chlorobenzyl)-5-methoxy-2-methyl-1H-indol-3-yl]-2-oxo-N-[3-(trifluoromethyl)phenyl]acetamide
2-[1-(4-chlorobenzyl)-5-methoxy-2-methyl-1H-indol-3-yl]-N-(2,6-difluorophenyl)-2-oxoacetamide
2-[1-(4-chlorobenzyl)-5-methoxy-2-methyl-1H-indol-3-yl]-N-(2-ethoxypyridin-4-yl)-2-oxoacetamide
2-[1-(4-chlorobenzyl)-5-methoxy-2-methyl-1H-indol-3-yl]-N-(2-fluoropyridin-4-yl)-2-oxoacetamide
2-[1-(4-chlorobenzyl)-5-methoxy-2-methyl-1H-indol-3-yl]-N-(2-methoxypyridin-4-yl)-2-oxoacetamide
2-[1-(4-chlorobenzyl)-5-methoxy-2-methyl-1H-indol-3-yl]-N-(3-chloro-4-fluorophenyl)-2-oxoacetamide
2-[1-(4-chlorobenzyl)-5-methoxy-2-methyl-1H-indol-3-yl]-N-(3-ethoxyphenyl)-2-oxoacetamide
2-[1-(4-chlorobenzyl)-5-methoxy-2-methyl-1H-indol-3-yl]-N-(3-ethylphenyl)-2-oxoacetamide
2-[1-(4-chlorobenzyl)-5-methoxy-2-methyl-1H-indol-3-yl]-N-(3-fluoropyridin-4-yl)-2-oxoacetamide
2-[1-(4-chlorobenzyl)-5-methoxy-2-methyl-1H-indol-3-yl]-N-(3-methylphenyl)-2-oxoacetamide
2-[1-(4-chlorobenzyl)-5-methoxy-2-methyl-1H-indol-3-yl]-N-(4-methoxypyridin-2-yl)-2-oxoacetamide
2-[1-(4-chlorobenzyl)-5-methoxy-2-methyl-1H-indol-3-yl]-N-(5-methoxypyridin-2-yl)-2-oxoacetamide
2-[1-(4-chlorobenzyl)-5-methoxy-2-methyl-1H-indol-3-yl]-N-(6-ethoxypyridin-3-yl)-2-oxoacetamide
2-[1-(4-chlorobenzyl)-5-methoxy-2-methyl-1H-indol-3-yl]-N-(6-methoxypyridin-2-yl)-2-oxoacetamide
2-[1-(4-chlorobenzyl)-5-methoxy-2-methyl-1H-indol-3-yl]-N-(6-methoxypyridin-3-yl)-2-oxoacetamide
2-[1-(4-fluorobenzyl)-2,5-dimethyl-1H-indol-3-yl]-N-(2-methoxypyridin-4-yl)-2-oxoacetamide
2-[1-(4-fluorobenzyl)-2-methyl-1H-indol-3-yl]-N-(2-methoxypyridin-4-yl)-2-oxoacetamide
2-[1-(4-fluorobenzyl)-5-methoxy-2-methyl-1H-indol-3-yl]-N-(2-methoxypyridin-4-yl)-2-oxoacetamide
2-[1-(4-fluorobenzyl)-5-methoxy-2-methyl-1H-indol-3-yl]-N-(3-fluorophenyl)-2-oxoacetamide
2-[2-chloro-1-(4-chlorobenzyl)-5-methoxy-1H-indol-3-yl]-N-(2-methoxypyridin-4-yl)-2-oxoacetamide
2-[2-chloro-1-(4-chlorobenzyl)-5-methyl-1H-indol-3-yl]-2-oxo-N-pyridin-4-ylacetamide
2-[2-chloro-1-(4-chlorobenzyl)-5-methyl-1H-indol-3-yl]-2-oxo-N-pyrimidin-4-ylacetamide
2-[2-chloro-1-(4-chlorobenzyl)-5-methyl-1H-indol-3-yl]-N-(2-chloropyridin-4-yl)-2-oxoacetamide
2-[2-chloro-1-(4-chlorobenzyl)-5-methyl-1H-indol-3-yl]-N-(2-methoxypyridin-4-yl)-2-oxoacetamide
2-[2-chloro-1-(4-chlorobenzyl)-5-methyl-1H-indol-3-yl]-N-(3-chlorophenyl)-2-oxoacetamide
2-[2-chloro-1-(4-chlorobenzyl)-5-methyl-1H-indol-3-yl]-N-(3-fluorophenyl)-2-oxoacetamide
2-[2-chloro-1-(4-chlorobenzyl)-5-methyl-1H-indol-3-yl]-N-(3-methoxyphenyl)-2-oxoacetamide
2-[2-chloro-1-(4-fluorobenzyl)-5-methoxy-1H-indol-3-yl]-2-oxo-N-pyridin-4-ylacetamide
2-[2-chloro-1-(4-fluorobenzyl)-5-methoxy-1H-indol-3-yl]-2-oxo-N-pyrimidin-4-ylacetamide
2-[2-chloro-1-(4-fluorobenzyl)-5-methoxy-1H-indol-3-yl]-N-(2-chloropyridin-4-yl)-2-oxoacetamide
2-[2-chloro-1-(4-fluorobenzyl)-5-methoxy-1H-indol-3-yl]-N-(2-methoxypyridin-4-yl)-2-oxoacetamide
2-[2-chloro-1-(4-fluorobenzyl)-5-methoxy-1H-indol-3-yl]-N-(3-chlorophenyl)-2-oxoacetamide
2-[2-chloro-1-(4-fluorobenzyl)-5-methoxy-1H-indol-3-yl]-N-(3-fluorophenyl)-2-oxoacetamide
2-[2-chloro-1-(4-fluorobenzyl)-5-methoxy-1H-indol-3-yl]-N-(3-methoxyphenyl)-2-oxoacetamide
2-[5-chloro-1-(4-chlorobenzyl)-2-methyl-1H-indol-3-yl]-N-(2-methoxypyridin-4-yl)-2-oxoacetamide
2-[5-chloro-1-(4-fluorobenzyl)-2-methyl-1H-indol-3-yl]-N-(2-methoxypyridin-4-yl)-2-oxoacetamide
2-[5-fluoro-1-(4-fluorobenzyl)-2-methyl-1H-indol-3-yl]-N-(2-methoxypyridin-4-yl)-2-oxoacetamide
2-[5-methoxy-1-(4-methoxybenzyl)-2-methyl-1H-indol-3-yl]-N-(2-methoxypyridin-4-yl)-2-oxoacetamide
2-[5-methoxy-2-methyl-1-(4-methylbenzyl)-1H-indol-3-yl]-N-(2-methoxypyridin-4-yl)-2-oxoacetamide
2-{5-methoxy-2-methyl-1-[4-(trifluoromethoxy)benzyl]-1H-indol-3-yl}-N-(2-methoxypyridin-4-yl)-2-oxoacetamide
2-{5-methoxy-2-methyl-1-[4-(trifluoromethyl)benzyl]-1H-indol-3-yl}-N-(2-methoxypyridin-4-yl)-2-oxoacetamide
N-(2-chloropyridin-4-yl)-2-[1-(2,4-dichlorobenzyl)-5-methoxy-2-methyl-1H-indol-3-yl]-2-oxoacetamide
N-(2-chloropyridin-4-yl)-2-[1-(4-fluorobenzyl)-5-methoxy-2-methyl-1H-indol-3-yl]-2-oxoacetamide
N-(2-chloropyridin-4-yl)-2-[5-methoxy-1-(4-methoxybenzyl)-2-methyl-1H-indol-3-yl]-2-oxoacetamide
N-(3-chlorophenyl)-2-[1-(4-fluorobenzyl)-5-methoxy-2-methyl-1H-indol-3-yl]-2-oxoacetamide
N-(3-chlorophenyl)-2-[5-methoxy-1-(4-methoxybenzyl)-2-methyl-1H-indol-3-yl]-2-oxoacetamide
N-(3-fluorophenyl)-2-[5-methoxy-1-(4-methoxybenzyl)-2-methyl-1H-indol-3-yl]-2-oxoacetamide.

In some embodiments of the above methods and uses, the FAAH inhibitor interacts with a TRPV1 receptor, a CB1 receptor, a PPARα receptor, a GPR119 receptor, a GPR55 receptor.

In some embodiments of the above methods and uses, the FAAH inhibitor is administered before a symptom of a sleep disorder, DDS and/or ICD develops in said patient. In further embodiments, the FAAH inhibitor is administered prior to, at the same time or after the initiation of dopaminergic agent treatment.

In some embodiments of the above methods and uses, the FAAH inhibitor is administered after one or more symptoms of a sleep disorder, DDS and/or ICD develop in said patient.

In some embodiments of the above methods and uses, the dopaminergic agent and the FAAH inhibitor are administered simultaneously. In other embodiments of the above methods and uses, the dopaminergic agent and the FAAH inhibitor are administered sequentially or separately.

In some embodiments, the above methods further comprise administering an additional therapeutic agent. In some embodiments, the pharmaceutical compositions and kits comprise an additional therapeutic agent.

In further embodiments, the additional therapeutic agent is a catechol O-methyltransferase (COMT) inhibitor, an anti-dyskinetic agent, an antipsychotic medication, a treatment for orthostatic hypotension, an agent to extend duration of anti-parkinsonian action, a monoamine oxidase B (MAO B) inhibitor, an anticholinergic medication, an antidepressant, an additional PD agent, or a combination thereof.

In yet further embodiments,
(a) said COMT inhibitor is entacapone (Comtan®), tolcapone (Tasmar®) or Stalevo® (combination of entacapone, levodopa and carbidopa);
(b) said anti-dyskinetic agent is amantadine (Symmetrel); fipamezole, sarizotan or saletracetam;
(c) said antipsychotic medication is clozapine (Clozaril®), ziprasidone (Geodon®), risperidone (Risperdal®), quetiapine (Seroquel®), olanzapine (Zyprexa®) or ACP-103;
(d) said monoamine oxidase B (MAO B) inhibitor is selegiline (Eldepryl®, Atapryl®, Carbex®) or rasagiline (Azilect®);
(e) said anticholinergic medication is trihexyphenidyl or benztropine (Cogentin);
(f) said antidepressant is amitriptyline (Elavil);
(g) said additional PD agent is Coenzyme Q10, an anti-apoptotic drug (e.g., CEP 1347 and CTCT346), an adenosine A2A receptor antagonist (e.g., istradefylline or preladenant) or an 5HT1A/5HT1B agonist (e.g., eltoprazine); and
(h) said treatment for orthostatic hypotension is L-DOPS (L-threo-dihydroxyphenylserine, Droxidopa®), fludrocortisone, midodrine, pidolol or clonidine.

In some embodiments, the pharmaceutical composition comprises (a) a FAAH inhibitor as discussed above, a pharmaceutically acceptable salt thereof, a pharmaceutically acceptable solvate (e.g., hydrate) or co-crystal of the compound or salt thereof, and (b) a pharmaceutically acceptable carrier, vehicle or adjuvant. In some embodiments, the pharmaceutical composition or kit comprises (a) a dopaminergic agent as discussed above, a pharmaceutically acceptable salt thereof, a pharmaceutically acceptable solvate (e.g., hydrate) or co-crystal of the compound or salt thereof, and (b) a pharmaceutically acceptable carrier, vehicle or adjuvant. In some embodiments, the pharmaceutical composition comprises (i) a FAAH inhibitor as discussed above, or a pharmaceutically acceptable salt thereof, (ii) a dopaminergic agent as discussed above, or a pharmaceutically acceptable salt thereof, and (iii) a pharmaceutically acceptable carrier, vehicle or adjuvant. In a further embodiment, the pharmaceutical composition further comprises at least one additional therapeutic agent.

### Pharmaceutically Acceptable Salts, Co-Forms and Pro-Drugs

In some embodiments of the methods or uses, the FAAH inhibitor may be provided as (i) the compound itself (e.g., as the free base); (ii) a pharmaceutically acceptable salt of the compound; (iii) a pharmaceutically acceptable solvate (e.g., hydrate) or co-crystal of the FAAH inhibitor compound or salt thereof; or (iv) part of a pharmaceutical composition. In some embodiments, the dopaminergic agent may be provided as (i) the compound itself (e.g., as the free base); (ii) a pharmaceutically acceptable salt of the compound; (iii) a pharmaceutically acceptable solvate (e.g., hydrate) or co-crystal of the dopaminergic agent or salt thereof; or (iv) part of a pharmaceutical composition.

The phrase “pharmaceutically acceptable salt,” as used herein, refers to pharmaceutically acceptable organic or inorganic salts of a compound described herein. For use in medicine, the salts of the compounds described herein will be pharmaceutically acceptable salts. Other salts may, however, be useful in the preparation of the compounds described herein or of their pharmaceutically acceptable salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

Pharmaceutically acceptable salts of the compounds described herein include those derived from suitable inorganic and organic acids and bases. In some embodiments, the salts can be prepared in situ during the final isolation and purification of the compounds. In other embodiments the salts can be prepared from the free form of the compound in a separate synthetic step.

When the compound described herein is acidic or contains a sufficiently acidic bioisostere, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particular embodiments include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound described herein is basic or contains a sufficiently basic bioisostere, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particular embodiments include citric, hydrobromic, hydrochloric, maleic, phosphoric, sulfuric and tartaric acids. Other example salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

The preparation of the pharmaceutically acceptable salts described above and other typical pharmaceutically acceptable salts is more fully described by Berg et al., “Pharmaceutical Salts,” J. Pharm. Sci., 1977:66:1-19, incorporated here by reference in its entirety.

In addition to the compounds described herein and their pharmaceutically acceptable salts, pharmaceutically acceptable solvates (e.g., hydrates) and co-crystals of these compounds and salts may also be employed in compositions to treat or prevent the herein identified disorders.

As used herein, the term “pharmaceutically acceptable solvate,” is a solvate formed from the association of one or more pharmaceutically acceptable solvent molecules to one of the compounds described herein. As used herein, the term “hydrate” means a compound described herein or a salt thereof that further includes a stoichiometric or non-stoichiometric amount of water bound by non-covalent intermolecular forces. The term solvate includes hydrates (e.g., hemihydrate, monohydrate, dihydrate, trihydrate, tetrahydrate, and the like).

“Pharmaceutically acceptable co-crystals” result when a pharmaceutically active compound crystallizes with another material (e.g., a carboxylic acid, a 4,4′-bipyridine or an excipient) that is also a solid at room temperature. Some pharmaceutically acceptable excipients are described in the next section. Other pharmaceutically acceptable substances that can be used to form co-crystals are given, for example by the GRAS (Generally Regarded As Safe) and the EAFUS (Everything Added to Food in the U.S.) databases maintained by the U.S. Food and Drug Administration (F.D.A.).

### Pharmaceutical Compositions and Methods of Administration

The compounds herein disclosed, and their pharmaceutically acceptable salts, solvates, co-crystals and pro-drugs thereof may be formulated as pharmaceutical compositions or “formulations.”

A typical formulation is prepared by mixing a compound described herein, or a pharmaceutically acceptable salt, solvate, co-crystal or pro-drug thereof, and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound described herein is being formulated. Solvents are generally selected based on solvents recognized by persons skilled in the art as safe (e.g., listed in the GRAS database) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include other types of excipients such as one or more buffers, stabilizing agents, antiadherents, surfactants, wetting agents, lubricating agents, emulsifiers, binders, suspending agents, disintegrants, fillers, sorbents, coatings (e.g., enteric or slow release) preservatives, antioxidants, opaquing agents, glidants, processing aids, colorants, sweeteners, perfuming agents, flavoring agents and other known additives to provide an elegant presentation of the drug (e.g., one or more of the compounds described herein or pharmaceutical composition thereof) or aid in the manufacturing of the pharmaceutical product (i.e., medicament).

The formulations may be prepared using conventional dissolution and mixing procedures. For example, the bulk drug substance (e.g., a compound described herein, a pharmaceutically acceptable salt, solvate, co-crystal or pro-drug thereof, or a stabilized form of the compound, such as a complex with a cyclodextrin derivative or other known complexation agent) is dissolved in a suitable solvent in the presence of one or more of the excipients described above. A compound having the desired degree of purity is optionally mixed with pharmaceutically acceptable diluents, carriers, excipients or stabilizers, in the form of a lyophilized formulation, milled powder, or an aqueous solution. Formulation may be conducted by mixing at ambient temperature at the appropriate pH, and at the desired degree of purity, with physiologically acceptable carriers. The pH of the formulation depends mainly on the particular use and the concentration of compound, but may range from about 3 to about 8.

A compound described herein or a pharmaceutically acceptable salt, solvate, co-crystal or pro-drug thereof is typically formulated into pharmaceutical dosage forms to provide an easily controllable dosage of the drug and to enable patient compliance with the prescribed regimen. Pharmaceutical formulations of compounds described herein, or a pharmaceutically acceptable salt, solvate, co-crystal or pro-drug thereof, may be prepared for various routes and types of administration. Various dosage forms may exist for the same compound. The amount of active ingredient that may be combined with the carrier material to produce a single dosage form will vary depending upon the subject treated and the particular mode of administration. For example, a time-release formulation intended for oral administration to humans may contain approximately 1 to 1000 mg of active material compounded with an appropriate and convenient amount of carrier material which may vary from about 5 to about 95% of the total composition (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

The pharmaceutical compositions described herein will be formulated, dosed, and administered in a fashion, i.e., amounts, concentrations, schedules, course, vehicles, and route of administration, consistent with good medical practice. Factors for consideration in this context include the particular disorder being treated, the particular human or other mammal being treated, the clinical condition of the individual patient, the cause of the disorder, the site of delivery of the agent, the method of administration, the scheduling of administration, and other factors known to medical practitioners, such as the age, weight, and response of the individual patient.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. The therapeutically effective amount of the compound to be administered will be governed by such considerations and is the minimum amount necessary to ameliorate, cure or treat the disease or disorder or one or more of its symptoms.

In some embodiments, a therapeutically effective amount of a dopaminergic agent for PD is one that ameliorates or alleviates the tremor of the hands, arms, legs, jaw, and face; rigidity or stiffness of the limbs and trunk; bradykinesia or slowness of movement; and postural instability or impaired balance and coordination caused by PD in a patient. In some embodiments, a therapeutically effective amount of a dopaminergic agent for RLS is one that ameliorates or alleviates the paresthesias (abnormal sensations) or dysesthesias (unpleasant abnormal sensations) in a patient with RLS. In some embodiments, a therapeutically effective amount of a dopaminergic agent for PLMD is one that ameliorates or alleviates the involuntary periodic limb jerking in a patient with PLMD.

In some embodiments, a therapeutically effective amount of a FAAH inhibitor is one that ameliorates or alleviates a sleep disorder, DDS or ICD. In further embodiments, a therapeutically effective amount of a FAAH inhibitor is one that ameliorates or alleviates one or more DDS, such as punding, stereotypies, hobbyism and/or walkabouts. In further embodiments, a therapeutically effective amount of a FAAH inhibitor is one that ameliorates or alleviates one or more ICD, such as pathological gambling, an eating disorder (e.g., excessive eating or binging), compulsive shopping and/or hypersexuality. In further embodiments, a therapeutically effective amount of a FAAH inhibitor is one that ameliorates or alleviates one or more sleep disorders, such as restlessness, insomnia, vivid dreaming and/or nightmares.

The term “prophylactically effective amount” refers to an amount effective in preventing or substantially lessening the chances of acquiring a disorder or in reducing the severity of the disorder or one or more of its symptoms before it is acquired or before the symptoms develop.

In some embodiments, a prophylactically effective amount of a FAAH inhibitor is one that prevents the occurrence or reoccurrence of a sleep disorder, DDS or ICD. In further embodiments, a prophylactically effective amount of a FAAH inhibitor is one that prevents the occurrence or reoccurrence of DDS, such as punding, stereotypies, hobbyism and/or walkabouts. In further embodiments, a prophylactically effective amount of a FAAH inhibitor is one that prevents the occurrence or reoccurrence of an ICD, such as pathological gambling, an eating disorder (e.g., excessive eating or binging), compulsive shopping and/or hypersexuality. In further embodiments, a prophylactically effective amount of a FAAH inhibitor is one that prevents the occurrence of a sleep disorder, such as restlessness, insomnia, vivid dreaming and/or nightmares.

Acceptable diluents, carriers, excipients, and stabilizers are those that are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, e.g., hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's: The Science and Practice of Pharmacy, 21^{st} Edition, University of the Sciences in Philadelphia, Eds., 2005 (hereafter “Remington's”).

“Controlled drug delivery systems” supply the drug to the body in a manner precisely controlled to suit the drug and the conditions being treated. The primary aim is to achieve a therapeutic drug concentration at the site of action for the desired duration of time. The term “controlled release” is often used to refer to a variety of methods that modify release of drug from a dosage form. This term includes preparations labeled as “extended release”, “delayed release”, “modified release” or “sustained release”.

“Sustained-release preparations” are the most common applications of controlled release. Suitable examples of sustained-release preparations include semipermeable matrices of solid hydrophobic polymers containing the compound, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers, and poly-D-(−)-3-hydroxybutyric acid.

“Immediate-release preparations” may also be prepared. The objective of these formulations is to get the drug into the bloodstream and to the site of action as rapidly as possible. For instance, for rapid dissolution, most tablets are designed to undergo rapid disintegration to granules and subsequent disaggregation to fine particles. This provides a larger surface area exposed to the dissolution medium, resulting in a faster dissolution rate.

Implantable devices coated with a compound of this invention are another embodiment of the present invention. The compounds may also be coated on implantable medical devices, such as beads, or co-formulated with a polymer or other molecule, to provide a “drug depot”, thus permitting the drug to be released over a longer time period than administration of an aqueous solution of the drug. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition.

The formulations include those suitable for the administration routes detailed herein. The formulations may conveniently be presented in unit dosage form and may be prepared by any of the methods well known in the art of pharmacy. Techniques and formulations generally are found in Remington's. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

The terms “administer”, “administering” or “administration” in reference to a compound, composition or formulation of the invention means introducing the compound into the system of the animal in need of treatment. When a compound of the invention is provided in combination with one or more other active agents, “administration” and its variants are each understood to include concurrent and/or sequential introduction of the compound and the other active agents.

The compositions described herein may be administered systemically or locally, e.g.: orally (e.g., using capsules, powders, solutions, suspensions, tablets, sublingual tablets and the like), by inhalation (e.g., with an aerosol, gas, inhaler, nebulizer or the like), to the ear (e.g., using ear drops), topically (e.g., using creams, gels, liniments, lotions, ointments, pastes, transdermal patches, etc), ophthalmically (e.g., with eye drops, ophthalmic gels, ophthalmic ointments), rectally (e.g., using enemas or suppositories), nasally, buccally, vaginally (e.g., using douches, intrauterine devices, vaginal suppositories, vaginal rings or tablets, etc), via an implanted reservoir or the like, or parenterally depending on the severity and type of the disease being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. In particular embodiments, the compositions are administered orally, intraperitoneally or intravenously.

The pharmaceutical compositions described herein may be orally administered in any orally acceptable dosage form including, but not limited to, capsules, tablets, aqueous suspensions or solutions. Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution-retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. Tablets may be uncoated or may be coated by known techniques including microencapsulation to mask an unpleasant taste or to delay disintegration and adsorption in the gastrointestinal tract and thereby provide a sustained action over a longer period. For example, a time-delay material such as glyceryl monostearate or glyceryl distearate alone or with a wax may be employed. A water-soluble taste-masking material such as hydroxypropyl-methylcellulose or hydroxypropyl-cellulose may be employed.

Formulations of a compound described herein that are suitable for oral administration may be prepared as discrete units such as tablets, pills, troches, lozenges, aqueous or oil suspensions, dispersible powders or granules, emulsions, hard or soft capsules, e.g., gelatin capsules, syrups or elixirs. Formulations of a compound intended for oral use may be prepared according to any method known to the art for the manufacture of pharmaceutical compositions.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with a water-soluble carrier such as polyethyleneglycol or an oil medium, for example peanut oil, liquid paraffin, or olive oil.

The active compounds can also be in microencapsulated form with one or more excipients as noted above.

When aqueous suspensions are required for oral use, the active ingredient is combined with emulsifying and suspending agents. If desired, certain sweetening and/or flavoring agents may be added. Syrups and elixirs may be formulated with sweetening agents, for example glycerol, propylene glycol, sorbitol or sucrose. Such formulations may also contain a demulcent, a preservative, flavoring and coloring agents and antioxidant.

Sterile injectable forms of the compositions described herein (e.g., for parenteral administration) may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of injectable formulations.

Oily suspensions may be formulated by suspending a compound described herein in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents, such as those set forth above, and flavoring agents may be added to provide a palatable oral preparation. These compositions may be preserved by the addition of an anti-oxidant such as butylated hydroxyanisol or alpha-tocopherol.

Aqueous suspensions of compounds described herein contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

The injectable formulations can be sterilized, for example, by filtration through a bacteria-retaining filter, or by incorporating sterilizing agents in the form of sterile solid compositions that can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable drug-depot forms are made by forming microencapsuled matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Drug-depot injectable formulations are also prepared by entrapping the compound in liposomes or microemulsions that are compatible with body tissues.

The injectable solutions or microemulsions may be introduced into a patient's bloodstream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

Compositions for rectal or vaginal administration are preferably suppositories which can be prepared by mixing the compounds described herein with suitable non-irritating excipients or carriers such as cocoa butter, beeswax, polyethylene glycol or a suppository wax which are solid at ambient temperature but liquid at body temperature and therefore melt in the rectum or vaginal cavity and release the active compound. Other formulations suitable for vaginal administration may be presented as pessaries, tampons, creams, gels, pastes, foams or sprays.

The pharmaceutical compositions described herein may also be administered topically, especially when the target of treatment includes areas or organs readily accessible by topical application, including diseases of the eye, the ear, the skin, or the lower intestinal tract. Suitable topical formulations are readily prepared for each of these areas or organs.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl ester wax, cetearyl alcohol, 2-octyldodecanol, benzyl alcohol and water.

For ophthalmic use, the pharmaceutical compositions may be formulated as micronized suspensions in isotonic, pH-adjusted sterile saline, or, preferably, as solutions in isotonic, pH-adjusted sterile saline, either with or without a preservative such as benzylalkonium chloride. Alternatively, for ophthalmic uses, the pharmaceutical compositions may be formulated in an ointment such as petrolatum. For treatment of the eye or other external tissues, e.g., mouth and skin, the formulations may be applied as a topical ointment or cream containing the active ingredient(s) in an amount of, for example, 0.075% to 20% w/w. When formulated in an ointment, the active ingredients may be employed with either an oil-based, paraffinic or a water-miscible ointment base.

Alternatively, the active ingredients may be formulated in a cream with an oil-in-water cream base. If desired, the aqueous phase of the cream base may include a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of emulsions prepared using compounds described herein may be constituted from known ingredients in a known manner. While the phase may comprise merely an emulsifier (otherwise known as an emulgent), it desirably comprises a mixture of at least one emulsifier with a fat or an oil or with both a fat and an oil. A hydrophilic emulsifier may be included together with a lipophilic emulsifier which acts as a stabilizer. In some embodiments, the emulsifier includes both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulgents and emulsion stabilizers suitable for use in the formulation of compounds described herein include Tween™-60, Span™-80, cetostearyl alcohol, benzyl alcohol, myristyl alcohol, glyceryl mono-stearate and sodium lauryl sulfate.

The pharmaceutical compositions may also be administered by nasal aerosol or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. Formulations suitable for intrapulmonary or nasal administration may have a mean particle size for example in the range of 0.1 to 500 microns (including particles with a mean size ranging between 0.1 and 500 microns in increments such as 0.5, 1, 30, 35 microns, etc.) and may be administered by rapid inhalation through the nasal passage or by inhalation through the mouth so as to reach the alveolar sacs.

The pharmaceutical composition (or formulation) for use may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

In another aspect, a compound described herein or a pharmaceutically acceptable salt, co-crystal, solvate or pro-drug thereof may be formulated in a veterinary composition comprising a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Therapeutic Methods

As used herein, the terms “subject” and “patient” are used interchangeably. The terms “subject” and “patient” refer to an animal (e.g., a bird such as a chicken, quail or turkey, or a mammal), preferably a “mammal” including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

“Treat”, “treating” or “treatment” with regard to a disorder or disease refers to alleviating or abrogating the effects of the disorder or disease. In some embodiments, the terms “treat”, “treatment” and “treating” as it pertains to the symptomatic treatment of PD refers to ameliorating or alleviating the tremor of the hands, arms, legs, jaw, and face, the rigidity or stiffness of the limbs and trunk, the bradykinesia or slowness of movement; and the postural instability or impaired balance and coordination caused by PD in a patient. In some embodiments, the terms “treat”, “treatment” and “treating” as it pertains to the symptomatic treatment of RLS refers to ameliorating or alleviating the paresthesias (abnormal sensations) or dysesthesias (unpleasant abnormal sensations) in a patient with RLS. In some embodiments, the terms “treat”, “treatment” and “treating” as it pertains to the symptomatic treatment of PLMD refers to ameliorating or alleviating the involuntary periodic limb jerking in a patient with PLMD.

In some embodiments, the terms “treat”, “treatment” and “treating” as it pertains to the use of a FAAH inhibitor refers to ameliorating or alleviating a sleeping disorder, DDS or ICD in a patient that exhibits these symptoms when being treated with a dopaminergic agent. In further embodiments, the terms “treat”, “treatment” and “treating” as it pertains to the use of a FAAH inhibitor refers to ameliorating or alleviating DDS such as punding, stereotypies, hobbyism and/or walkabouts. In further embodiments, the terms “treat”, “treatment” and “treating” as it pertains to the use of a FAAH inhibitor refers to ameliorating or alleviating an ICD, such as pathological gambling, an eating disorder (e.g., excessive eating or binging), compulsive shopping and/or hypersexuality. In further embodiments, the terms “treat”, “treatment” and “treating” as it pertains to the use of a FAAH inhibitor refers to ameliorating or alleviating a sleep disorder, such as restlessness, insomnia, vivid dreaming and/or nightmares. In still further embodiments, the terms “treat”, “treatment” and “treating” as it pertains to the use of a FAAH inhibitor refers to ameliorating or alleviating one or more of a sleeping disorder, ICD and DDS.

In some embodiments, the terms “prevent”, “prevention” and “preventing” as it pertains to the use of a FAAH inhibitor refers to inhibiting the occurrence or reoccurrence of DDS or ICD in a patient who is being treated with a dopaminergic agent. In further embodiments, the terms “prevent”, “prevention” and “preventing” as it pertains to the use of a FAAH inhibitor refers to inhibiting the occurrence or reoccurrence of DDS, such as punding, stereotypies, hobbyism and/or walkabouts. In further embodiments, the terms “prevent”, “prevention” and “preventing” as it pertains to the use of a FAAH inhibitor refers to inhibiting the occurrence or reoccurrence of an ICD, such as pathological gambling, an eating disorder (e.g., excessive eating or binging), compulsive shopping and/or hypersexuality. In further embodiments, the terms “prevent”, “prevention” and “preventing” as it pertains to the use of a FAAH inhibitor refers to inhibiting the occurrence or reoccurrence of an a sleep disorder, such as restlessness, insomnia, vivid dreaming and/or nightmares. In still further embodiments, the terms “prevent”, “prevention” and “preventing” as it pertains to the use of a FAAH inhibitor refers to inhibiting the occurrence or reoccurrence of one or more of a sleep disorder, ICD and DDS. As used herein, the terms “prevent”, “prevention” and “preventing” refer to the reduction in the risk of acquiring or developing a sleep disorder, DDS or an ICD, or the reduction or inhibition of the recurrence or said condition in a patient.

Compounds and compositions of the invention are also useful for veterinary treatment of companion animals, exotic animals and farm animals, including, without limitation, dogs, cats, mice, rats, hamsters, gerbils, guinea pigs, rabbits, horses, pigs and cattle.

### Combination Therapies

The compounds and pharmaceutical compositions described herein can be used in combination therapy with one or more additional therapeutic agents. For combination treatment with more than one active agent, where the active agents are in separate dosage formulations, the active agents may be administered separately or in conjunction. In addition, the administration of one agent may be prior to, concurrent to, or subsequent to the administration of the other agent.

When co-administered with other agents, e.g., when co-administered with another pain medication, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, compounds described herein can be administered to a subject in a dosage range from between about 0.001 to about 100 mg/kg body weight/day, from about 0.001 to about 50 mg/kg body weight/day, from about 0.001 to about 30 mg/kg body weight/day, from about 0.001 to about 10 mg/kg body weight/day.

When “combination therapy” is employed, an effective amount can be achieved using a first amount of a compound described herein or a pharmaceutically acceptable salt, solvate (e.g., hydrate), co-crystal or pro-drug thereof and a second amount of an additional suitable therapeutic agent (e.g., an agent to treat pain).

In one embodiment of this invention, the compound described herein and the additional therapeutic agent are each administered in an effective amount (i.e., each in an amount which would be therapeutically effective if administered alone). In another embodiment, the compound described herein and the additional therapeutic agent, are each administered in an amount which alone does not provide a therapeutic effect (a sub-therapeutic dose). In yet another embodiment, the compound described herein can be administered in an effective amount, while the additional therapeutic agent is administered in a sub-therapeutic dose. In still another embodiment, the compound described herein can be administered in a sub-therapeutic dose, while the additional therapeutic agent, for example, a suitable cancer-therapeutic agent is administered in an effective amount.

As used herein, the terms “in combination” or “co-administration” can be used interchangeably to refer to the use of more than one therapy (e.g., one or more prophylactic and/or therapeutic agents). The use of the terms does not restrict the order in which a therapy (e.g., prophylactic and/or therapeutic agents) is administered to a subject.

Co-administration encompasses administration of the first and second amounts of the compounds in an essentially simultaneous manner, such as in a single pharmaceutical composition, for example, capsule or tablet having a fixed ratio of first and second amounts, or in multiple, separate capsules or tablets for each. In addition, such co-administration also encompasses use of each compound in a sequential manner in either order. When co-administration involves the separate administration of the first amount of a compound described herein and a second amount of an additional therapeutic agent, the compounds are administered sufficiently close in time to have the desired therapeutic effect. For example, the period of time between each administration that can result in the desired therapeutic effect can range from minutes to hours and can be determined taking into account the properties of each compound such as potency, solubility, bioavailability, plasma half-life and kinetic profile. For example, a compound described herein and the second therapeutic agent can be administered in any order within about 24 hours of each other, within about 16 hours of each other, within about 8 hours of each other, within about 4 hours of each other, within about 1 hour of each other or within about 30 minutes of each other.

More, specifically, a first therapy (e.g., a prophylactic or therapeutic agent such as a compound described herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks prior to), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks subsequent to) the administration of a second therapy (e.g., a dopaminergic agent or other PD treatment agent) to a subject.

### EXAMPLES

Examples 1-9 show the ability of an example FAAH inhibitor, URB597, to reduce hyperactive behavioral L-DOPA side effects in MPTP-lesioned primate (marmoset and macaque) subjects while maintaining the anti-parkinsonian benefits of the L-DOPA treatment.

All references provided in the Examples are herein incorporated by reference in their entirety. As used herein, all abbreviations, symbols and conventions are consistent with those used in the contemporary scientific literature. See, e.g., Janet S. Dodd, ed., The ACS Style Guide: A Manual for Authors and Editors, 2^{nd} Ed., Washington, D.C.: American Chemical Society, 1997, herein incorporated by reference in its entirety.

### Example 1

### Induction of Parkinsonian Syndrome in Marmosets

Six common marmosets (Callithrix jacchus; female; weight, 350-500 g (Harlan, Madison, Wis., USA)) were kept under conditions of controlled temperature (25±2° C.) and a 12-hour light/dark cycle (lights on 6:30 a.m.). Animals were group housed and had access to food, fresh-fruit supplements and water ad libitum and their home cage environment was enriched with primate toys, perches and auditory stimuli. Prior to start of studies, animals were acclimatized to handling, blood collection and administration of treatments (oral syringe feeding and subcutaneous injection) as well as transfer to observation cages for assessment of behavior. A parkinsonian syndrome was induced by once-daily subcutaneous administration of MPTP hydrochloride (Sigma, Canada; 2 mg/kg base concentration in 0.9% saline) for 5 consecutive days. Following this, animals were allowed to recover for 12 weeks to allow parkinsonian symptoms to develop and stabilize. MPTP intoxication resulted in a syndrome characterized by bradykinesia, hunched posture and a reduced range of movement. Treatment-related complications including dyskinesia and psychosis-like behaviors were evoked by twice-daily treatment with L-DOPA p.o. (as Prolopa®, Roche Canada, Ontario, equivalent to 15 mg/kg L-DOPA and 3.5 mg/kg benserazide) for a minimum of 30 days. This treatment regimen has been previously demonstrated to produce a stable model of L-DOPA-induced complications [Gomez-Ramirez et al. (2006) Mov. Disord. 21:839-846; Visanji et al. (2008) Mov. Disord. 23:1922-1925; Visanji et al (2009) Neurobiol Dis. 35: 184-192]. After this time, the animals were used in several studies to evaluate potential anti-parkinsonian and anti-dyskinetic agents. A minimum period of two weeks was allowed between completing any prior study and commencing this study, a period in excess of ten half-lives since administration of any previously employed experimental drug. Throughout the course of the study, on days not requiring plasma sampling or behavioral observations, animals were treated once daily with oral L-DOPA therapy, given at 10:00 a.m. to maintain stable levels of complications. Marmosets treated with MPTP as described in this example are referred to as MPTP-lesioned marmosets.

### Example 2

### Data Presentation, Statistical Analysis, Drugs and Formulation

### Data Presentation and Statistical Analysis

To assess the effect of treatment on plasma FAAH substrate level, total activity, high activity time and count, data were analyzed using a parametric repeated-measures (RM) 1-way analysis of variance (ANOVA) followed by a Student Newman-Keuls Multiple Comparison post hoc test. To assess the effect of treatment on plasma L-DOPA level a paired, two-tailed t-test was used. All FAAH (AEA, OEA, PEA) subbstrate and L-DOPA plasma level data and activity data are presented as the group mean and SEM For measures of parkinsonian disability, dyskinesia and psychosis, data were analyzed via Friedman's test with Dunn's Multiple Comparison post hoc test. Disability data are displayed as the median, with individual values (on graphs) or interquartile range (IQR) in text. In all cases, p<0.05 was taken to represent a significant difference.

### Drugs and Formulation

MPTP hydrochloride (Sigma, Canada) was dissolved in 0.9% NaCl sterile solution to a concentration of 0.2 mg/ml (freebase) and administered at 1 ml/kg. Maintenance L-DOPA, administered as Prolopa® capsules (Roche, Mississauga, ON, Canada) containing 50 mg L-DOPA and 12.5 mg benserazide (freebase) was dissolved in Gatorade sports beverage (The Gatorade Company, Chicago, Ill.) prior to oral administration by syringe at a volume of 10 ml/kg. On days of behavioral assessment, L-DOPA (20 mg/kg, freebase) was prepared from the methyl ester form (Sigma, Canada) in combination with benserazide hydrochloride (5 mg/kg, freebase, Sigma, Canada) dissolved in 0.9% sterile saline containing 0.1% ascorbate (Sigma, Canada) and 0.05% absolute ethanol. URB597 (Cayman Chemical, Ann Arbor, Mich.) was formulated in a vehicle containing 50% corn oil (Professional Compounding Centers of America (PCCA), Houston, Tex.) and 50% nutritional drink Ensure™ (Abbott Nutrition, Chicago, Ill.) to make the suspension palatable for marmoset oral dosing. Requisite amounts of URB597 were weighed into sterile 50 ml polypropylene tubes (for the 3, 10, 30 and 60 mg/kg doses, 12, 40, 120 and 240 mg were weighed respectively). Then, 10 ml Corn oil was added and mixed until homogenous. Next, 10 ml Ensure™ was added and the mixture mixed gently at first, vortexed and finally sonicated (Ultrasonic Dismembrator, Fisher Scientific model 100, ⅛″) on a medium setting for 30 sec to ensure homogeneity of the suspension. URB597 or vehicle was dosed orally at a volume of 5 ml/kg body weight.

### Example 3

### Assessment of Plasma FAAH Substrate Levels in MPTP-Lesioned Marmosets in Response to Treatment with URB597

### Treatments

The effect of oral treatment of MPTP-lesioned marmosets with either vehicle or URB597 (3, 10, 30 or 60 mg/kg) on plasma levels of specific endogenous FAAH substrates was assessed. Levels of the endocannabinoid AEA, in addition to the non-cannabinoid fatty acid amides N-palmitoyl-ethanolamide (PEA) and N-oleoyl-ethanolamide (OEA), were measured by LC-MS/MS assays (described below). At 4:00 p.m. on days prior to plasma sampling, animals were orally administered either vehicle (described below) or URB597 (3, 10, 30 or 60 mg/kg). Doses were given in a non-randomized fashion, being administered in ascending order of dose. At 7:00 a.m. the following day, all food was removed from the animals' cages and, at 10:00 a.m. on the day of sampling, animals were again treated with the same dose of URB597 or vehicle. Four hours later, at 2:00 p.m., under light isofluorane anesthetic (Forane®, Baxter Healthcare, Canada), 1 ml of blood was drawn from the great saphenous vein using a 27G butterfly needle and collected into 2×0.5 ml K₂-EDTA-containing vials (BD microtainer®, BD, Mississauga, Canada). Plasma was prepared within 10 min of collection by centrifugation of blood at 13,000×g for 10 min at room temperature. Plasma was then transferred to sterile 1.5 ml tubes prior to immediate freezing on dry ice and subsequent storage at −80° C.

### Bioanalytical Assessment of AEA, OEA and PEA in MPTP-Lesioned Marmoset Plasma

The concentrations of endogenous AEA, OEA and PEA levels in plasma were determined by LC-MS/MS using d-4-AEA, d-4-OEA and d-4-PEA stable isotope-labeled surrogate calibrators, with d8-AEA added as an internal standard (Cayman Chemicals, Ann Arbor, Mich.). The fatty acid amides were extracted from 200 μl plasma samples and standards by protein precipitation extraction with three volumes of chilled chloroform:methanol (1:2, v:v), followed by liquid-liquid extraction with chloroform. After evaporation under nitrogen, the extracts were reconstituted in 60 μl of acetonitrile/isopropanol/water (20:5:75, v:v:v). The samples were injected (20 μl) on a Clipeus C8 HPLC column (2.1 mm×30 mm dimensions; 5 μm particle size; Higgins Analytical, Mountain View, Calif.) and chromatographed under reverse phase conditions, using a gradient system with 0.1% formic acid in water and 0.1% formic acid in acetonitrile/isopropanol/water (85:10:5, v:v:v). The compounds were detected and quantified by tandem mass spectrometry in positive ion mode on an API 4000 (Applied Biosystems; Framingham, Mass.). The limit of quantization for all three analytes was 0.3 ng/ml.

### URB597 Elevates Plasma Levels of AEA, OEA and PEA in MPTP-Lesioned Marmosets

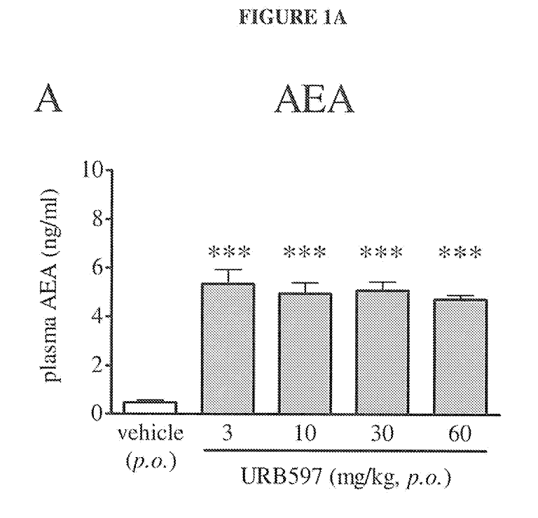

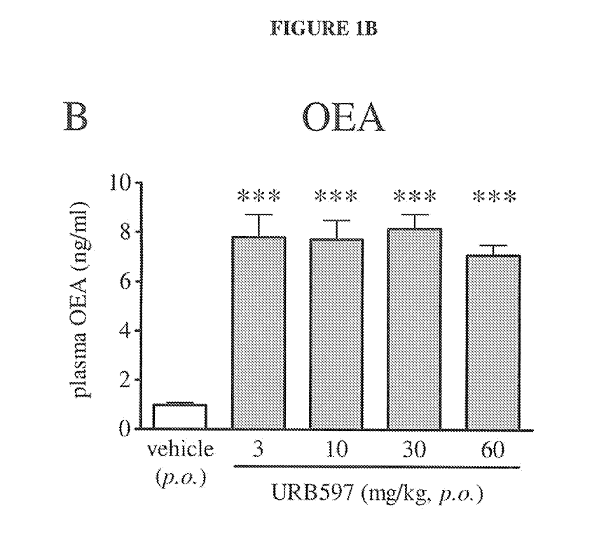

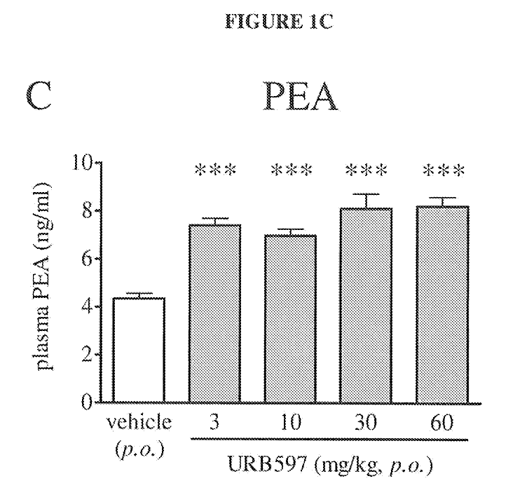

FAAH inhibitor URB597 elevated plasma levels of three FAAH substrates (AEA, OEA, and PEA) assessed in MPTP-lesioned marmosets (AEA, F_{4,20}=47.8; OEA, F_{4,20}=32.7; and PEA, F_{4,20}=15.2, RM-ANOVA). Student Newman-Keuls analysis revealed a significant increase in plasma levels of all three FAAH substrates assessed compared to placebo treatment (all p<0.001, FIGS. 1A-C respectively). Four hours after oral administration, mean plasma levels of AEA, OEA and PEA were 0.49±0.09, 0.98±0.10 and 4.34±0.21 ng/ml in vehicle-treated monkeys and were 5.0±0.45, 7.7±0.78 and 7.0±0.27 ng/ml, respectively, in URB597 (10 mg/kg)-treated monkeys (FIGS. 1A-C). All four doses of URB597 administered (from 3 mg/kg to 60 mg/kg) showed similar fatty acid amide elevations (i.e., no dose response) which were not significantly different from each other (Student Newman-Keuls post-hoc analysis, all p>0.05) suggesting complete inhibition of FAAH at all dose levels studied. The observed mean pharmacodynamic effects of URB597 treatment of the 4 doses studied each compared to vehicle treatment on plasma levels of AEA, OEA, PEA were 10.3±0.3, 7.8±0.2 and 1.8±0.1, respectively (mean fold change of URB597 groups ±SEM). The 10 mg/kg dose of URB597 was chosen for use in behavioral studies.

### Example 4

### The Effect of URB597 on Plasma L-DOPA Levels in MPTP-Lesioned Marmosets

### Treatments

The effect of oral treatment with either vehicle or URB597 (10 mg/kg) on plasma levels of L-DOPA was assessed at the beginning of the period of peak L-DOPA effect, i.e., 2 h after L-DOPA administration. At 4:00 p.m. on days prior to plasma sampling, animals were treated orally with either vehicle or URB597 (10 mg/kg). On days of plasma sampling, animals were fed their normal diet between 7:00-7:30 a.m. after which time all food was removed from their cages. Water was available ad libitum. At approximately 9:00 a.m., each animal received either vehicle (p.o.) or URB597 (p.o.). Two hours after this, at approximately 11:00 a.m., animals received either vehicle or L-DOPA (s.c., 20 mg/kg) treatment. Two hours later, at 2:00 p.m., animals were lightly anaesthetized as described above and 1 ml of blood was removed from the great saphenous vein using a 27G butterfly needle and collected into 2×0.5 ml K₂-EDTA-containing vials (BD microtainer®, BD, Mississauga, Canada,). Plasma was prepared within 10 min of collection by centrifugation of blood at 13,000×g for 10 min at room temperature. Plasma was then transferred to sterile 1.5 ml tubes prior to immediate freezing on dry ice and subsequent storage at −80° C.

### Bioanalytical Assessment of L-DOPA Levels

The concentrations of L-DOPA in marmoset plasma samples were determined using LC-MS/MS by modification of a published procedure for quantification of L-DOPA in human plasma samples (Li et al. 2000, J Pharm. Biomed. Anal. 24: 325-333). Plasma samples were transferred between laboratories on dry ice and then stored at −80° C. until analysis. A standard curve of L-DOPA was prepared in marmoset plasma using a 0.5 mg/ml stock solution of L-DOPA (Sigma, St. Louis, Mo.) in water.

Plasma samples from MPTP-lesioned marmosets that had been treated orally with vehicle or URB597 and injected subcutaneously with L-DOPA and benserazide were thawed at room temperature and a 100 μl aliquot taken from each sample. To each aliquot, 400 μl of ice cold acetonitrile containing 100 ng of deuterium-labeled L-DOPA [L-DOPA-d₃ internal standard 3-(3,4-Dihydroxyphenyl-2,5,6-d3)-L-alanine obtained from Sigma, St. Louis Mo.] was added. The individual plasma and acetonitrile samples were centrifuged at 15,000×g for 10 min at room temperature. The supernatant was collected and dried under a stream of nitrogen. The dried pellet was resuspended in 2% acetonitrile in water containing 0.1% formic acid.

Ten μl of each sample was injected onto a Waters Atlantis T3 column (2 mm×100 mm dimensions, 3 micron particle size) on a Waters Acquity HPLC system (Waters Corporation, Milford, Mass.). L-DOPA and L-DOPA-d3 were detected as they eluted from the column using a mobile phase gradient over 1.7 min beginning with 2% acetonitrile in water and increasing to 20% acetonitrile in water. The mobile phase water contained 0.1% formic acid. Detection of L-DOPA and L-DOPA-d3 was conducted using a Waters TQD mass spectrometer (Waters Corporation, Milford, Mass.) operating in MRM mode. The lower limit of quantification of L-DOPA by this LC-MS/MS method (or lowest usable standard for this assay) in plasma from MPTP-lesioned marmosets was 5 ng/ml.

URB597 does not Alter Plasma L-DOPA Levels

URB597 did not alter plasma L-DOPA levels at the time of peak L-DOPA action (2 h post administration of L-DOPA, 4 h post administration of URB597, p>0.05, paired t-test; see Table 1).

TABLE 1
Effect of URB597 treatment on plasma L-DOPA levels
in MPTP-lesioned marmosets

| | | plasma L-DOPA |
|---|---|---|
| ^{a}Treatment 1 | ^{b}Treatment 2 | concentration (ng/ml) |
| vehicle (p.o.) | L-DOPA (20 mg/kg, s.c.) | 1618 ± 135^{c} |
| URB597 (10 mg/kg, p.o.) | L-DOPA (20 mg/kg, s.c.) | 1543 ± 162 |

n = 6 per treatment group, data are mean ± SEM.
^{a}URB597 or vehicle administered twice at 4:00 p.m. on day prior to sample and at 9:00 a.m. on day of sample
^{b}L-DOPA contains benserazide (5 mg/kg) given at 11:00 a.m. on day of sample
^{c}vehicle/L-DOPA cf. URB597/L-DOPA, p > 0.05, paired t-test.

### Example 5

### Behavioral Effect of URB597 Alone or in Combination with L-DOPA in MPTP-Lesioned Marmosets

The effects of URB597 (10 mg/kg, p.o.) alone or in combination with L-DOPA (20 mg/kg, s.c.) on motor activity, parkinsonian disability, dyskinesia and psychosis were assessed in a group of MPTP-lesioned marmosets (n=6) with stable L-DOPA-induced dyskinesia. Based on previous dose-finding studies in these animals (data not shown) a dose of L-DOPA (20 mg/kg)/benserazide (5 mg/kg), was chosen such that it provided the best anti-parkinsonian effect achievable whilst eliciting hyperactivity, dyskinesia and psychosis that was stable and reproducible on successive L-DOPA administrations. For behavioral observations, L-DOPA was administered s.c. at a dose volume of 1 ml/kg, as L-DOPA methyl ester (Sigma, Canada) in combination with benserazide (Sigma, Canada). Based on its ability to maximally elevate plasma levels of AEA, PEA and OEA in MPTP-lesioned marmosets, a dose of 10 mg/kg URB597 was employed for all behavioral observations and administered orally at a dose volume of 5 ml/kg.

### Treatments

The effect of URB597 alone and on L-DOPA response was assessed. On days prior to behavioral assessment, animals were fed normally and received a maintenance oral L-DOPA dose at 9:00 a.m. At 4:00 p.m. animals were administered either vehicle (p.o.) or URB597 (10 mg/kg, p.o.). On days of behavioral assessment, animals were fed their normal diet between 7:00-7:30 a.m. after which time all food was removed from their cages. Water was available ad libitum. At approximately 9:00 a.m., each animal received either vehicle (p.o.) or URB597 (p.o.). Two hours after this, at approximately 11:00 a.m., animals received either vehicle or L-DOPA (s.c.) treatment. Behavioral assessment, as described below commenced directly following this second treatment.

To prevent any confounding effects of prior treatment with oral URB597 on the assessment of response to vehicle treatment, the order of these treatments was randomized in each animal. A minimum of 48 h was left between behavioral observations in the same.

### Behavioral Assessment of Marmoset Behavior

After administration of final treatment (L-DOPA, s.c.), animals were placed immediately into observation cages (0.8×0.8×0.7 m) containing food, water and a wooden perch and left undisturbed for the 6 h duration of the experiment. Behavior was monitored via recorded DVD footage and analyzed post hoc by a movement disorder neurologist blinded to the treatment. Methods for assessment of behavior were essentially as described previously (Fox et al., 2006, Arch. Neurol. 63: 1343-1344; Gomez-Ramirez et al 2006 Mov Disord. 21:839-846). Parkinsonian disability scores were assessed every 10 min for the duration of assessment. A parkinsonian disability rating scale combined measures of range of movement, bradykinesia, posture and alertness to allow a maximum score of 36 and cumulated further during the period of peak L-DOPA action (2-4 h after L-DOPA administration) giving a total maximum score of 432. Briefly, range of movements were rated on a scale of 0 to 9: 0=running, jumping between roof, walls, perch, uses limbs through a wide range of activity; 9=no movement. Bradykinesia was rated from 0 to 3: 0=normal initiation and speed of movement; 1=slight slowing of movement; 2=moderate slowing of movement, marked freezing, difficulty initiating and maintaining movement; 3=prolonged freezing, akinetic, inability to move. Postural abnormalities were rated 0 or 1: 0=normal balance, upright posture, head held up; 1=impaired balance, crouched posture, head down. Attention/alertness was rated 0 or 1; 0=normal head checking movements—movement of neck in variable directions—smooth, small movements; 1=reduced or absent head checking, head in one position for more than 50% of observation period. A global parkinsonian disability score was rated as a combination of the behaviors mentioned above according to the following formula: (range of movement×1)+(bradykinesia×3)+(posture×9)+(alertness×9).

L-DOPA-induced dyskinesia and psychosis were independently assessed during the same period of peak anti-parkinsonian action of L-DOPA (2-4 h after injection). During this period, for each 10 min epoch, dyskinesia and psychosis were rated from 0 to 4: 0=absent; 1=mild, fleeting, rare, present less than 30% of the observation period; 2=moderate, not interfering with normal activity, present more than 30% of the observation period; 3=marked, at times interfering with normal activity, present less than 70% of the observation period; 4=severe, continuous, replacing normal activity, present more than 70% of the observation period. For dyskinesia, chorea and dystonia were graded separately and the score given to represent the most disabling dyskinesia observed, whether chorea or dystonia, in any 10-minute period of assessment. For psychosis, hyperkinesia, response to non-apparent stimuli (hallucinatory behavior), repetitive grooming and stereotypies were graded separately. For this measure, the score given represented the most disabling of any of the four sub-score levels observed, in any 10 min period of assessment.

In addition, a quantitative assessment of marmoset activity was made using computer-operated passive infra-red sensors, essentially as described previously (Maccarrone et al., 2003, J. Neurochem. 85: 1018-1025; Visanji et al., 2009 J Pharm Exp Ther 328: 276-283). A single sensor containing a hemispherical lens (Guardall, Mississauga, ON, Canada) was mounted 1.5 m above the top of each the observation cage. The sensor was positioned so that motion was detected throughout the entirety of the cage below. The signal was fed via an RS-232 input to a computer. Proprietary Motion Detector software (Research Electronics, Toronto Western Hospital, Toronto, ON, Canada) was utilized that displayed within Microsoft Excel (Microsoft, Redmond, Wash.). Activity counts were logged in 1-min epochs for the entire 6 h duration of the experiment and cumulated over the peak dose period of 2-4 h.

Assessment of marmoset activity was further assessed over time by quantifying the average activity of counts per minute of the same animals obtained prior to administration of MPTP (i.e., in the normal state). Activity over the same period of 2-4 h was calculated and used to identify minutes of high activity (a minute when activity was above the average per minute of the animal prior to MPTP). High activity counts (the total counts obtained in high activity minutes) were cumulated. High activity time (the number of high activity minutes) were also calculated.

### URB597 Reduces L-DOPA-Induced Motor Activity in MPTP-Lesioned Marmosets

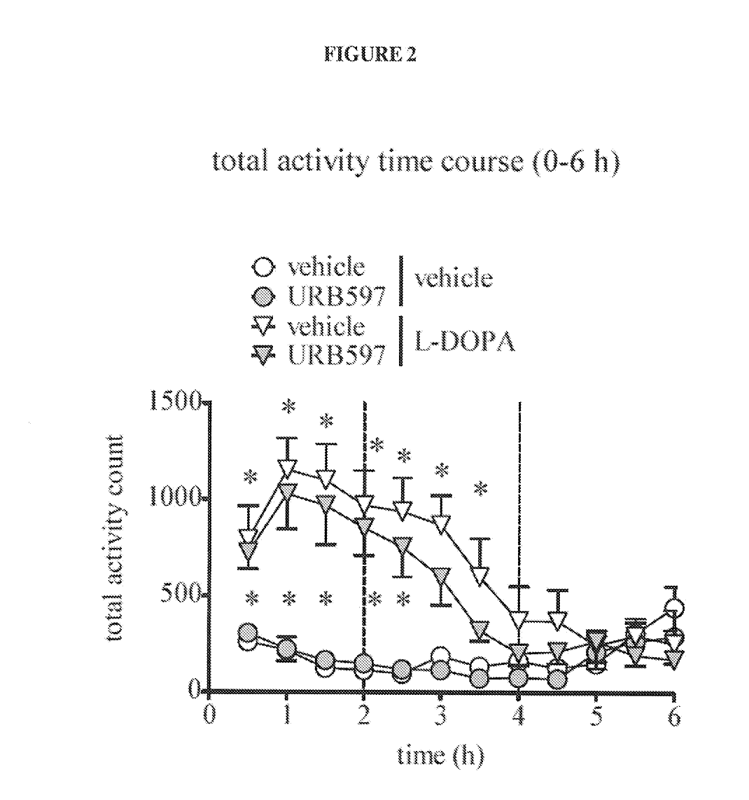

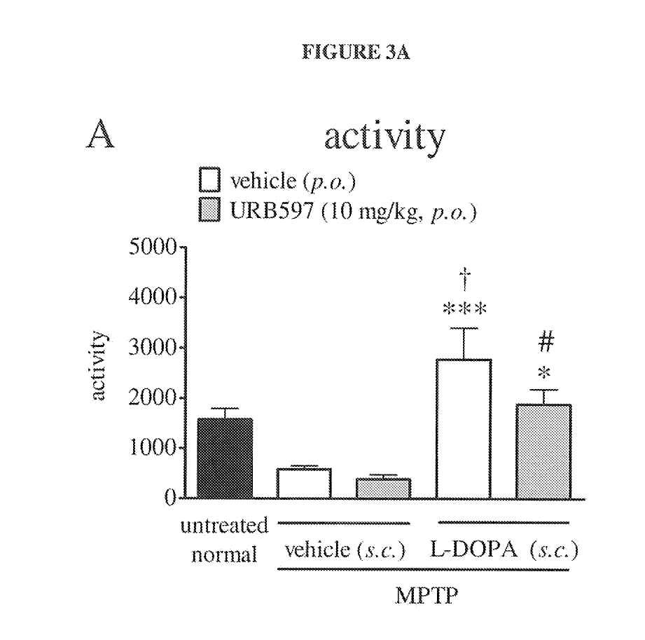

L-DOPA elicited a marked increase in total motor activity in MPTP-lesioned marmosets. Examination of the time-course of activity across the whole 6 h period of observation revealed a significant effect of time, treatment and the interaction of the two on total activity counts (F_{time 11,220}=11.96, F_{treatment 3,220}=18.65, and F_{interaction 33,220}=5.18, all p<0.001, two-way RM-ANOVA; see FIG. 2). Further analysis demonstrated that L-DOPA-induced total activity was significantly increased compared to that of vehicle (s.c.)-treated, MPTP-lesioned animals for the first 3.5 h following drug administration (all p<0.05). In comparison, URB597 (10 mg/kg, p.o.) elicited a reduction in the magnitude of L-DOPA-induced activity such that after 2.5 h counts were no longer significantly different compared to vehicle (s.c.)-treated, MPTP-lesioned animals (see FIG. 2). Subsequent analyses was conducted on the 2-4 h period post L-DOPA administration, when the effect of URB597 on L-DOPA-induced activity was maximal.

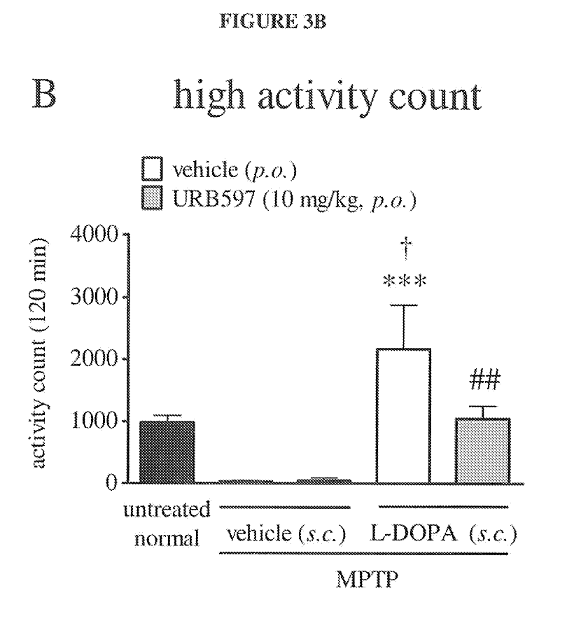

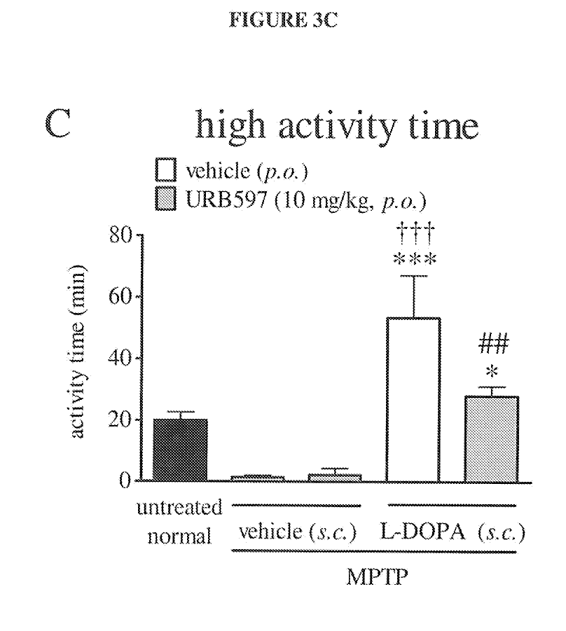

Analysis of total activity counts in the peak dose period revealed there was a significant effect of treatment on activity (F_{4,20}=8.47, p<0.001, RM-ANOVA). Further analysis demonstrated that L-DOPA-induced activity (FIG. 3A) was significantly increased (by 379%), compared to activity in vehicle (s.c.)-treated, MPTP-lesioned animals (2782±682 cf. 581±74 counts, p<0.001). Furthermore, L-DOPA induced a level of activity in MPTP-lesioned marmosets that was significantly increased, by 77%, compared to activity observed in non-treated, non-MPTP-lesioned (normal) animals (1572±235 counts, p<0.05). This hyperactivity is considered to represent a non-human primate correlate of dopamine dysregulation syndrome and impulse control disorder (Fox et al., 2006, Arch. Neurol. 63: 1343-1344). Treatment with URB597 (10 mg/kg, p.o.) attenuated L-DOPA-induced increases in activity to levels significantly lower (by 32%) than that seen in the presence of L-DOPA alone (1879±332 counts, p<0.05 cf. L-DOPA alone) and to a level similar to that seen in normal, un-lesioned animals (119% of normal, p>0.05).

In a similar fashion, there was also a significant effect of treatment on both levels of high activity counts and time spent in a state of high activity (high activity count, F_{5,20}=6.99; high activity time, F_{4,20}=10.3 both p<0.001, RM-ANOVA, FIGS. 3B and 3C). L-DOPA significantly increased high activity counts (from 31±17 to 2170±778 counts) and high activity time (from 1.3±0.8 to 53.3±15.2 min) compared to vehicle treatment in MPTP-lesioned marmosets (all p<0.001). In addition, MPTP-lesioned marmosets treated with L-DOPA showed a significant increase in high activity counts (by 120%, p<0.05) and high activity time (by 167%, p<0.001) as compared to normal, non-MPTP-lesioned animals (high activity counts; 985±118, high activity time; 20±2.9 min). In MPTP-lesioned marmosets, URB597 (10 mg/kg, p.o.) significantly reduced high activity counts (by 52%) and activity time (by 48%) evoked by L-DOPA (both p<0.05). High activity counts and high activity time were not significantly different to those seen in normal, unlesioned animals (p>0.05).

URB597 does not Interfere with the Anti-Parkinsonian Actions of L-DOPA in MPTP-Lesioned Marmosets

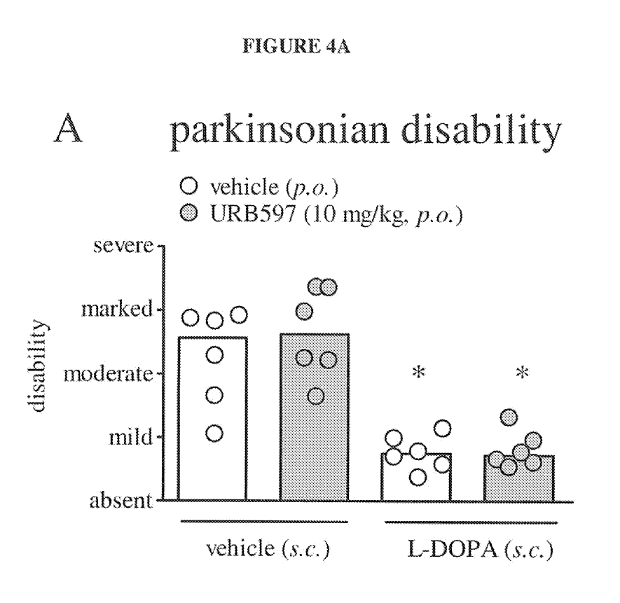

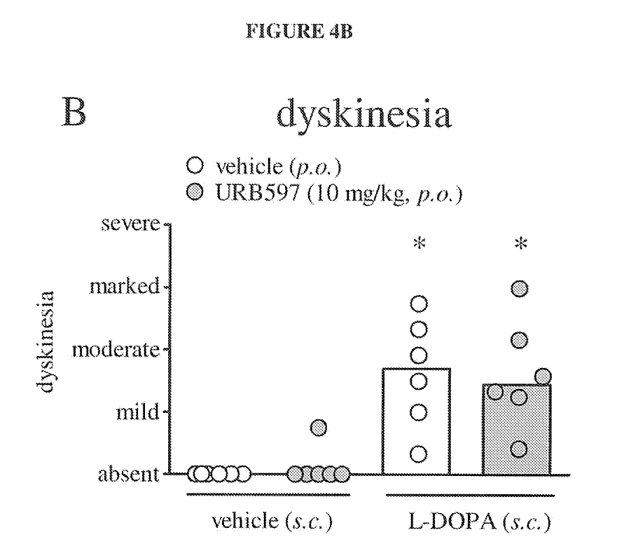

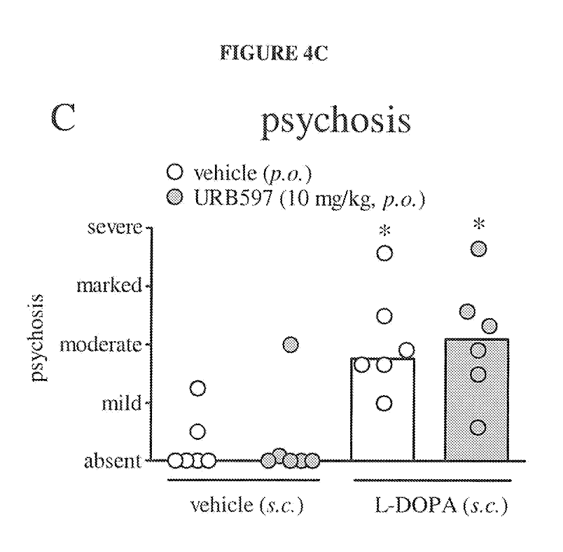

URB597 did not modify anti-parkinsonian actions of L-DOPA (FIG. 4A). The MPTP-lesioned animals used in the current study displayed a moderate to marked level of parkinsonian disability (median, 277; IQR, 163-312). At times of peak L-DOPA effect (2-4 h post administration), L-DOPA significantly attenuated the level of parkinsonian disability in MPTP-lesioned marmosets to a mild to absent level (median, 80; IQR, 57-111, Friedman Statistic (FS)=15.2, p<0.05 with Dunn's post hoc test). L-DOPA in combination with URB597 (10 mg/kg, p.o.) produced a similar significant alleviation of parkinsonism to mild to absent levels (median, 78; IQR, 65-114, p<0.05) that was not significantly different to that exhibited by animals treated with L-DOPA alone (p>0.05, Dunn's Multiple Comparison test).

URB597 does not Modify L-DOPA-Induced Dyskinesia in MPTP-Lesioned Marmosets

URB597 did not modify L-DOPA-induced dyskinesia (FIG. 4B). The MPTP-lesioned marmosets employed in the present study exhibited a mild to moderate level of dyskinesia, which was a combination of chorea and dystonia, following treatment with L-DOPA at times of peak L-DOPA action (2-4 h post administration; median, 20; IQR, 10-29, FS=15.9, p<0.05 with Dunn's post hoc test). The level of dyskinesia in MPTP-lesioned animals administered with L-DOPA in combination with URB597 (10 mg/kg, p.o.) was also mild to moderate (median, 18; IQR, 13-29) and, whilst significantly different to that seen with vehicle alone (median, 0; IQR, 0-0, p<0.05), was not significantly different to that seen when treated with L-DOPA alone (p>0.05, Dunn's Multiple Comparison test).

URB597 does not Modify L-DOPA-Induced Psychosis in the MPTP-Lesioned Marmoset

URB597 did not modify L-DOPA-induced psychosis (FIG. 4C). The parkinsonian animals used in the present study also exhibited a mild to moderate level of L-DOPA-induced psychosis following treatment with L-DOPA at peak L-DOPA action (2-4 h post administration) (median, 0; IQR, 0-0, FS=11.3, p<0.05). The level of psychosis in MPTP-lesioned animals administered with L-DOPA in conjunction with URB597 (10 mg/kg, p.o.) was also mild to moderate and whilst significantly greater than that seen with vehicle alone (p<0.05) was not significantly different to those animals treated with L-DOPA alone (p>0.05, Dunn's Multiple Comparison test).

### URB597 Monotherapy has No Effect on Behavior in the MPTP-Lesioned Marmoset

URB597 given alone did not modify total activity, high activity, parkinsonian disability or elicit dyskinesia or psychosis (Student Newman-Keuls post-hoc analysis, all p>0.05 cf. vehicle) (FIGS. 3 and 4).

### Example 6

### Behavioral Effects of Treatment Vehicle in Normal Macaques

Behavior in normal macaques was assessed following administration of URB597 vehicle (i.v.) and L-DOPA vehicle (p.o.) given to normal, non-MPTP-lesioned macaques. The data provided by this example established baseline levels of behavior, particularly activity, with which to compare against that seen in MPTP-lesioned animals. The experiment examined the behavioral responses of vehicle treatment (URB597 and L-DOPA vehicles, administered i.v. and p.o. respectively) in six normal macaques.

### Husbandry

Six cynomolgus monkeys (Macaca fascicularis) were used in each experiment. Animals were group housed with up to 3 animals per cage. The cage sizes exceeded UK, NIH, EU and CCAC minimum size recommendations, 2.4 m×3.0 m×1.0 m. The housing room was subject to a 12 hour light-dark cycle (lights on 7 a.m.), temperature 20-25° C. in a room containing only animals of the same sex. Fresh fruit, primate pellets and water was available ad libitum unless specifically described in other examples below.

For a period of 3 months before commencing treatment, animals were acclimatized to the experimental setting and trained to receive oral and intravenous administration of vehicle. During this period, animals were handled by technical staff and transferred from home caging to observation caging on a regular basis.

### Vehicle Treatment Regime to Assess Baseline Behavior in Normal (Unlesioned) Macaques

After acclimatization, the six macaques in the unlesioned group were administered water p.o. at a dose volume of 1 ml/kg which was the vehicle for Madopar™ treatments (L-DOPA in combination with benserazide in the ratio 4:1). The normal macaques were also administered the URB597 vehicle, which was 0.9% sterile saline containing 1% DMSO (v/v) (Sigma-Aldrich product #D2650) and 2% (v/v) Tween-80 (Sigma-Aldrich product #P4780) for dosing at a volume of 1 ml/kg. The URB597 vehicle was prepared by adding DMSO, Tween-80 and saline in that order to a sterile 50 ml Falcon™ tube and very gently vortex-mixing the tube contents after each addition.

On days prior to the day of behavioral assessment, the macaques were fed normally and not fasted. After their last feed of the day (˜4 p.m.) the macaques were not fed again until the start of behavioral observations the following day.

On days of behavioral assessment, the macaques were fasted until the start of behavioral observations at approximately 9:30 a.m. At approximately 9:00 a.m., each animal received URB 597 vehicle (i.v.). Thirty minutes later, the macaques received L-DOPA vehicle (p.o.) treatment. Behavioral assessment (6 h), as described in Example 5, commenced directly following this second treatment.

Blood samples (1 ml), were removed at approximately 30 min following completion of behavioral observations (4 p.m.) corresponding to ˜7 hours post URB vehicle administration. Each animal in turn was restrained and blood samples taken using a 25G butterfly needle attached to a 10 ml syringe. After collection, pressure was applied to the area to stem any bleeding. Samples were collected into 2×BD microtainer™ 0.5 ml-fill K₂EDTA tubes (two for each 1 ml of blood), gently inverted and centrifuged within 10 minutes of collection at 13,000×g for 10 min at room temperature. The uppermost plasma layer from each sample (minimum 0.4 ml each) was carefully removed and transferred to individual sterile 1.5 ml collection tubes. The plasma samples were then stored at −80° C. until required for further analysis as described in Example 3.

### Example 7

### Behavioral Effects of the FAAH Inhibitor, URB597, in Parkinsonian MPTP-Lesioned Macaques

This experiment examined the behavioral responses to five doses of URB597 (0.01, 0.03, 0.1, 0.3 or 1 mg/kg, i.v.) or vehicle in combination with L-DOPA in six parkinsonian macaques previously lesioned with MPTP.

### Treatment Regime to Assess Baseline Behavior in MPTP-Lesioned Macaques

The protocols for housing and acclimatization for the six parkinsonian MPTP-lesioned macaques were as those detailed in Example 6.

After acclimatization, the macaques in the MPTP cohort were rendered parkinsonian by once daily subcutaneous injection of 0.2 mg/kg MPTP, administered for 8-12 days, until the first appearance of parkinsonism symptoms. After this time, a parkinsonian syndrome evolved to a moderate to marked level, over approximately 30 days, and stabilized. Additional administrations of MPTP were given to some animals to titrate to similar degrees of parkinsonism in individuals across the group. The macaques were allowed to recover for a minimum of further 30 days until their parkinsonism was demonstrated as being stable.

Sixty days after commencing MPTP administration, L-DOPA (20 mg/kg) was administered orally twice daily for six months. L-DOPA was administered as Madopar™ (L-DOPA in combination with benserazide). This treatment led to the development of motor fluctuations, including dyskinesia and wearing-off.

Dose-finding studies were performed to define a dose of L-DOPA for each individual macaque that provided the best anti-parkinsonian effect achievable and elicited hyperactivity that was stable and reproducible for that animal, on successive L-DOPA administrations. A dose was chosen such that the hyperactive state, (i.e., defined by activity counts and activity time) significantly exceeded in magnitude that seen in an equivalent animal in the normal unlesioned state. The L-DOPA doses employed for behavioral observations had a mean (±s.e.m.) of 38.3±2.3 mg/kg, and ranged from 30 to 45 mg/kg.

L-DOPA was administered p.o. at a dose volume of 1 ml/kg, as Madopar™ (L-DOPA in combination with benserazide in the ratio 4:1). The vehicle for Madopar™ was water. On days when animals were not subject to behavioral observations they received maintenance oral L-DOPA, Madopar™ (30 mg/kg, p.o.) at approximately 9:00 a.m.

URB597 was obtained from Cayman Chemical (product #10046) and was formulated in 0.9% sterile saline containing 1% DMSO (v/v) (Sigma-Aldrich product #D2650) and 2% (v/v) Tween-80 (Sigma-Aldrich product #P4780) for dosing at a volume of 1 ml/kg.

For each URB597 concentration tested, requisite amounts of URB597 were weighed into sterile 50 ml sterile polypropylene tubes (Falcon™) (e.g., for the 1 mg/kg doses, approximately 35 mg was weighed). Next, 0.35 ml of DMSO was added and very gently vortexed until all particles were dissolved. Next, 0.7 ml Tween-80 was slowly added directly to the existing DMSO within the tube and again very slowly vortexed until homogenous. Maintaining a slow vortexing of the open tube, dropwise, the remaining 33.95 ml of 0.9% saline was added. The mixture remained clear with no trace of undissolved particles. Once all the saline was incorporated, the solution was syringe filtered using Whatman (GE Healthcare, NJ, USA) GD/X 25 mm sterile glass microfiber syringe filters with a 0.45 micron size particle retention rating. Once filtered, the solution was loaded into 5 ml syringes, one per animal, for dosing at a volume of 1 ml/kg.

The treatments that were assessed are given below in Table 2. URB597/vehicle or URB597 vehicle alone were administered i.v. L-DOPA/vehicle or L-DOPA vehicle alone were administered p.o.

TABLE 2
Treatments administered to each MPTP-lesioned macaque.

| treatment | animals | drug | dose | drug | |
|---|---|---|---|---|---|
| # | (n = 6) | i.v. | (mg/kg) | p.o. | procedure |
| 2 | MPTP | vehicle¹ | — | vehicle² | behavior |
| 3 | MPTP | vehicle¹ | — | L-DOPA | behavior |
| 4 | MPTP | URB597 | 0.01 | L-DOPA | behavior |
| 5 | MPTP | URB597 | 0.03 | L-DOPA | behavior |
| 6 | MPTP | URB597 | 0.1 | L-DOPA | behavior |
| 7 | MPTP | URB597 | 0.3 | L-DOPA | behavior |
| 8 | MPTP | URB597 | 1 | L-DOPA | behavior |

To prevent any potential long-term effects of URB597 treatment from influencing outcome, all animals received all treatments but in an ascending dose non-randomized design. A minimum of 72 h was left between behavioral observations in the same animals. During this period, animals were fed normally and not fasted. Animals also received their normal maintenance oral L-DOPA dose at 9:00 a.m.

On days prior to the day of behavioral assessment, the macaques were fed normally and not fasted and they received their normal maintenance oral L-DOPA dose at 9:00 a.m. After their last feed of the day (˜4 p.m.) animals were not fed again until start of behavioral observations the following day.

On days of behavioral assessment, animals were fasted until start of behavioral observations at approximately 9:30 a.m. At approximately 9:00 a.m., each animal received either vehicle (p.o.) or URB597 (URB, i.v.). Thirty minutes later, at approximately 9:30 a.m., animals then received either vehicle or L-DOPA (p.o.) treatment. Behavioral assessment (6 h), as described in Example 5, commenced directly following this second treatment. Blood samples were removed at approximately 30 min following completion of behavioral observations (4 p.m.) corresponding to ˜7 hours post URB597 administration to be analyzed for endogenous substrates of fatty acid amide hydrolase (FAAH) such as N-oleoyl ethanolamine (OEA), as detailed in Example 3.

URB597 Reduced L-DOPA Hyperactivity in MPTP-Lesioned Macaques, with the Most Pronounced Effect 2-3 Hours after L-DOPA Administration

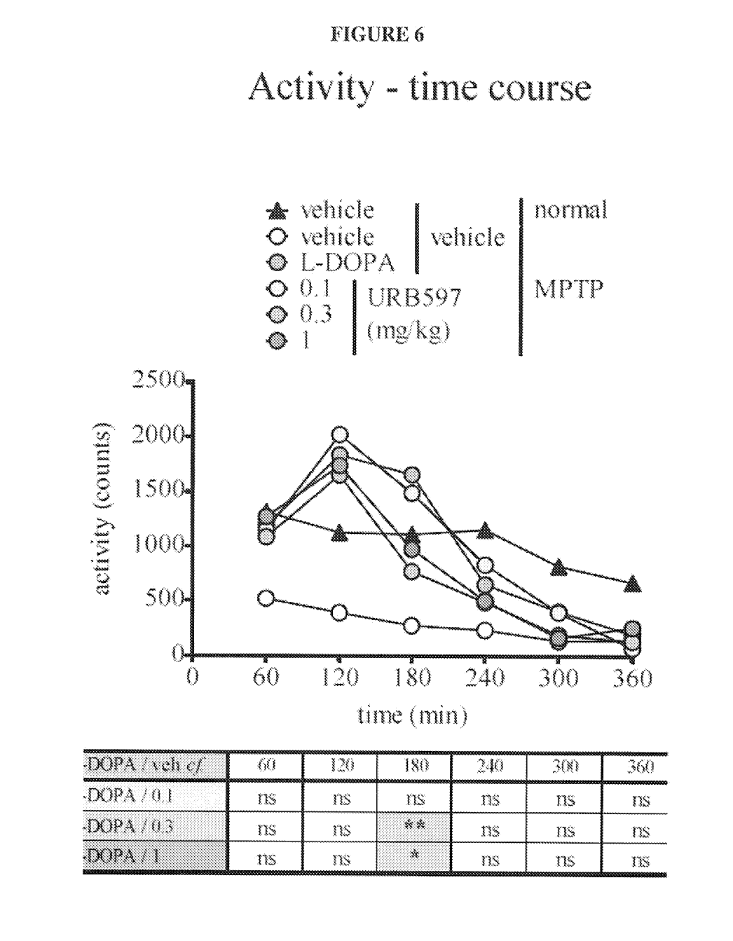

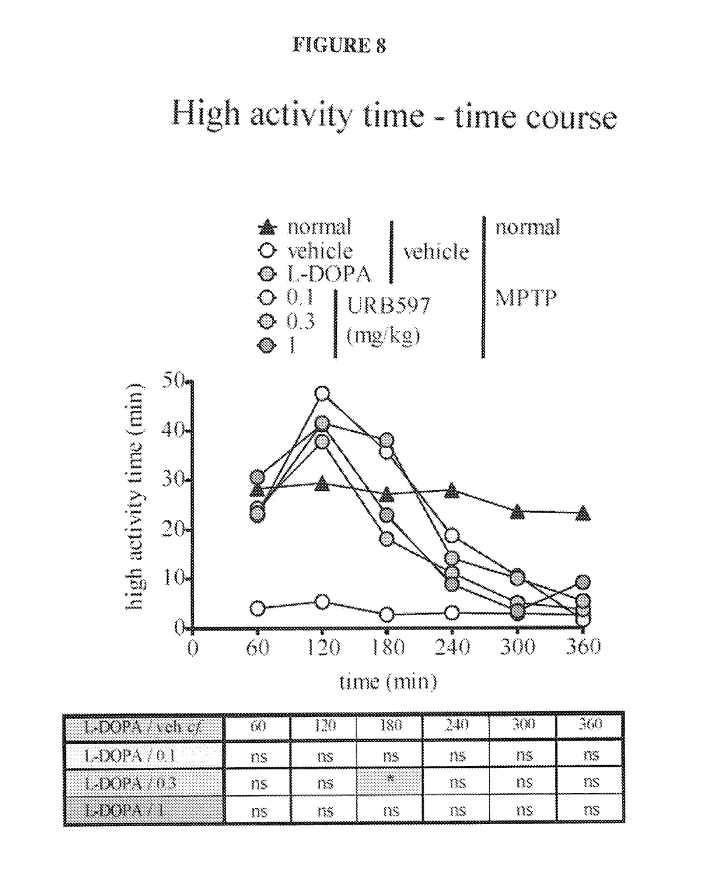

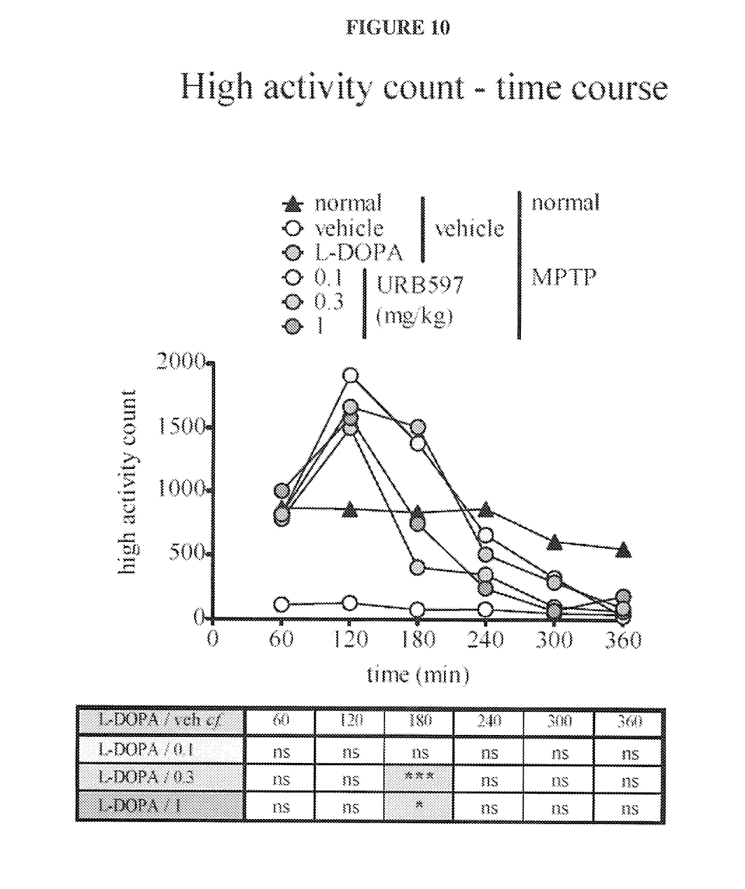

The basic outcome seen in Example 5 with the marmoset model was broadly reproduced in a macaque model. The total activity (FIG. 6), times of high activity (FIG. 8), and high activity count (FIG. 10) were assessed. As in Example 5, total activity counts, and high activity time and counts were higher in MPTP-lesioned macaques 1 to 3.5 hrs after administration of L-DOPA compared to normal, unlesioned macaques.

MPTP-lesioned macaques treated with 0.3 and 1.0 mg/ml URB597 and L-DOPA showed a significant reduction of hyperactive behavior compared with treatment with L-DOPA alone. A Repeated-Measures ANOVA was performed to account for the effects of time, treatment, and time-treatment interaction on the total activity, times of high activity and high activity count data across the whole 6 h period of observation. The analysis revealed a significant effect of UBR597 treatment versus L-DOPA treatment only (*/*** represent p<0.05 or p<0.001, respectively) at 3 hrs for: total activity for UBRB597 at the 0.3 mg and 1.0 mg/kg levels (FIG. 6); high activity time at 0.3 mg/kg level (FIG. 8); and 0.3 and 1.0 mg/kg on high activity count (FIG. 10).

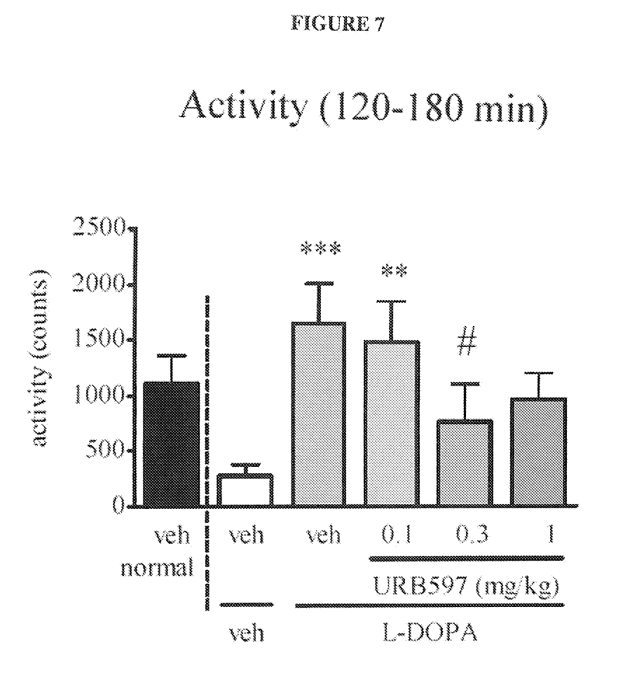

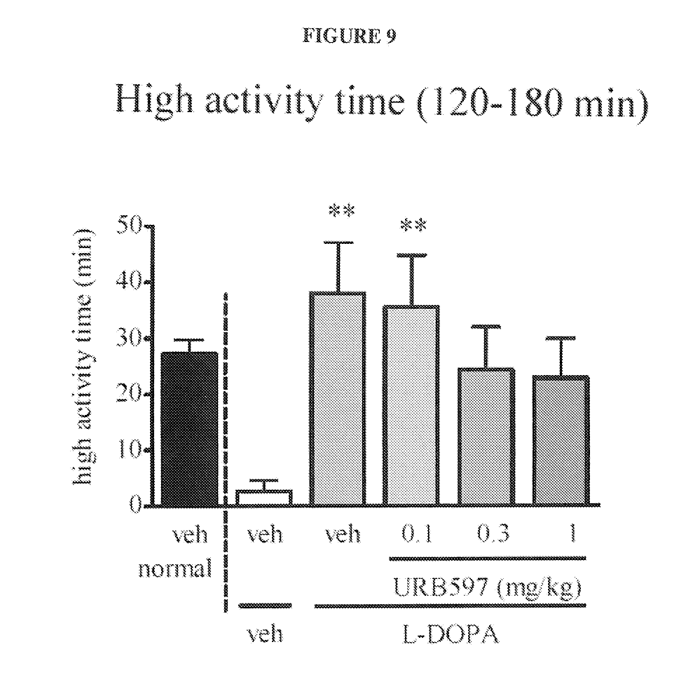

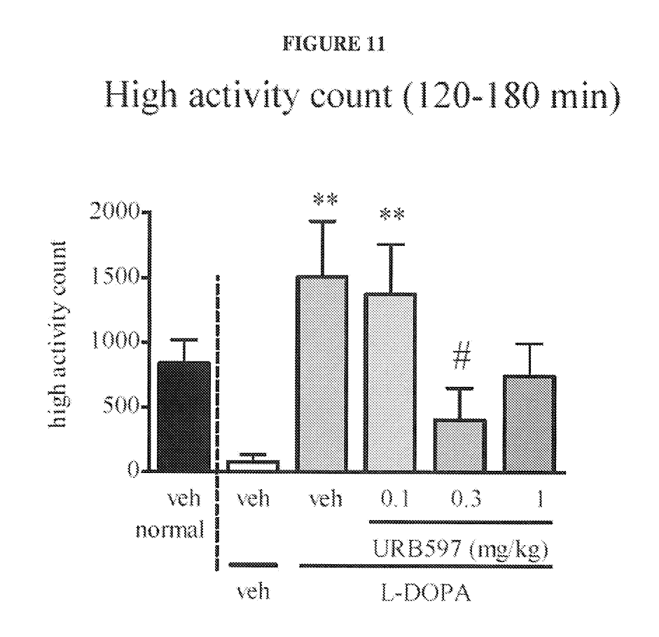

Further analysis of the total activity, times of high activity and high activity count data between 2 and 3 h after administration of L-DOPA, (FIGS. 7, 9, and 11), showed that animals treated with L-DOPA alone and L-DOPA/URB597 (0.1 ml/kg) had significantly higher total activity counts (FIG. 7), high activity time (FIG. 9), and high activity counts (FIG. 11) than normal macaques (**/*** represent p<0.01 or p<0.001, respectively, cf. vehicle (p.o.)/vehicle (s.c.) treated, MPTP-lesioned animals).

Between 2-3 h post administration of L-DOPA, the 0.3 mg/ml treatment showed significantly lower total activity (FIG. 7) and high activity count (FIG. 11) from the L-DOPA alone treatment (# represents p<0.05 cf. vehicle (p.o.)/L-DOPA (s.c.) treated, MPTP-lesioned animals).

These results suggest that URB597 dose-dependently reduced levels of L-DOPA-induced hyperactivity. At 3 hrs post-administration of L-DOPA, MPTP-lesioned macaques administered URB597 at the 0.3 and 1.0 mg/kg dose showed lowered hyperactivity over the L-DOPA treatments alone. The effect was most pronounced in the 2-3 h period following administration of L-DOPA, during which time L-DOPA alone still evokes a hyperactive response. In terms of dose effect, the dose response is very steep between 0.1 and 0.3 mg/kg, such that 0.1 mg/kg is ineffective, while URB597 at 0.3 mg/kg is maximally effective. Based on this outcome but subject to revision following completion of full behavioral analysis (parkinsonian disability, dyskinesia and psychosis) the 0.3 mg/kg dose will be employed in further experiments in the macaque model.

### Example 8

### Behavior Following Administration of URB597 (Vehicle, 0.1, 0.3 and 1 mg/kg, I.V.) in Combination with L-DOPA Vehicle in Normal Animals

To assess the effect of URB597 alone in normal animals, doses of URB597 may be administered in an ascending manner, along with the L-DOPA vehicle (water) as similarly administered in Example 6. Activity data is obtained and analysed in an on-going manner. Blood samples can be withdrawn and analyzed for fatty acid amides as described in Example 3.

As found in the marmoset model, the URB597 administration alone is expected to show elevated levels of AEA, OEA, and PEA, but have little or minor effect on behavior of unlesioned, normal macaques.

### Example 9

### Assessment of Plasma Levels of Fatty Acid Amides in Macaques Treated with 0.3 ml URB597/L-DOPA

Plasma levels of fatty acid amides can be assessed by treating a group of MPTP-lesioned macaques (n=6) with consecutive treatments of URB597 vehicle/L-DOPA vehicle, URB597 vehicle and L-DOPA, and 0.3 mg/kg URB597 and L-DOPA, with a minimum of one week between blood sampling in the same animal.

Husbandry and administration of the treatments (i.e., fasting protocol, etc.) to each macaque would follow the husbandry and administration protocol given in Example 6. Two blood samples can be taken per animal per treatment, one sample taken just prior to treatment, and one taken 24 hours after administration with the treatment.

The blood samples can be analyzed for endogenous substrates of FAAH and plasma L-DOPA levels. Macaques treated with URB597 are expected to reveal higher levels of endogenous substrate of FAAH and little to no change in plasma L-DOPA levels.

### Example 10

### Combination Treatment with URB597 and TRPV1 Antagonist Inhibitor

The behavioral responses to a single dose of URB597 (i.v.), 3 mg/kg, chosen on the basis of Experiment 1a, or vehicle, in combination with multiple doses of the TRPV1 antagonist SB366791 (s.c.), or its respective vehicle, in combination with L-DOPA in the six parkinsonian macaques previously lesioned with MPTP may be assessed as described below. Inhibition of the TRVP1 receptor has been reported to reduce dyskinesia in some animal models (van der Stelt and DiMarzo, 2004, Eur. J. Biochem. 271:1827-1834.)

### Treatments

Six cynomolgus monkeys (Macaca fascicularis) are to be used in this study, the same as employed in Example 6.

Doses of L-DOPA would follow those provided in Example 6. On days when animals are not subject to behavioral observations they would receive maintenance oral L-DOPA, Madopar™ (30 mg/kg, p.o.) at approximately 9:00 a.m.

URB597 is formulated in the same manner as described in Experiment 1a (section 3.2.3) 1% DMS (v/v) (Sigma-Aldrich product #02650, batch TBD and 2% (v/v) Tween-80 (Sigma-Aldrich product #P4780, batch TBD). Dosing volume would be maintained at 1 ml/kg. SB366791 can be formulated for subcutaneous injection in 0.9% sterile saline containing 10% (v/v) DMSO. Dosing volume would be 0.2 ml/kg.

Treatments assessed are given below in Table 3.

TABLE 3
Dosing regime for URB597/SB366791 combination study.

| | animals | drug¹ | dose | drug² | drug³ | dose | |
|---|---|---|---|---|---|---|---|
| treatment # | (n = 6) | i.v. | (mg/kg) | p.o. | s.c. | (mg/kg) | procedure |
| 16 | MPTP | URB597 | 0.3 | L-DOPA | SB366791 | 0.05 | behavior |
| 17 | MPTP | URB597 | 0.3 | L-DOPA | SB366791 | 0.1 | behavior |
| 18 | MPTP | URB597 | 0.3 | L-DOPA | SB366791 | 0.5 | behavior |

Analysis of behavior - dose selection of SB366791

| | | | | | | | |
|---|---|---|---|---|---|---|---|
| 19 | MPTP | URB597 | 0.3 | vehicle² | SB366791 | TBD* | behavior |

Washout of URB597

| | | | | | | | |
|---|---|---|---|---|---|---|---|
| 20 | MPTP | vehicle¹ | — | vehicle² | vehicle³ | — | behavior |
| 21 | MPTP | vehicle¹ | — | L-DOPA | vehicle³ | — | behavior |
| 22 | MPTP | vehicle¹ | — | vehicle² | SB366791 | TBD* | behavior |
| 23 | MPTP | vehicle¹ | — | L-DOPA | SB366791 | TBD* | behavior |

vehicle¹, vehicle used to formulate URB597 given i.v.
vehicle², vehicle used to formulate L-DOPA given p.o.
vehicle³, vehicle used to formulate SB366791 given s.c.
*dose of SB366791 to be chosen on the basis of effect of treatments 16-18.

To prevent any potential long-term effects of URB597 treatment from influencing outcome, all animals would receive all treatments but in a semi-randomized design. Treatments 16-18 are conducted first in randomized fashion using an incomplete Latin Square-type design. Once behavioral data are analyzed and, a dose of SB366791 selected, treatment 19 would then completed in a non-randomized, single-treatment design. Following this, a period of at least 1 week would be allowed for wash-out of URB597. Treatments 20-23 are then conducted with a randomized design using an incomplete Latin Square-type design. A minimum of 72 h would be left between behavioral observations in the same animals.

Hyperactivity, parkinsonian disability and dyskinesia is assessed as in the protocol detailed in Example 5. The combination treatment of UBS597 and SB366791 would be expected to show reductions in hyperactivity as well as dyskinesia over the same animals treated with L-DOPA only.

1-82. (canceled)

83. A method of treating or preventing an impulse control disorder (ICD), dopamine dysregulation syndrome (DDS) or sleep disorder arising from the treatment of a patient suffering from Parkinson's disease (PD), restless legs syndrome (RLS) or periodic limb movement disorder (PLMD) or combination thereof with a dopaminergic agent, comprising administering a therapeutically effective amount of a FAAH inhibitor, or a therapeutically effective amount of a dopaminergic agent and a therapeutically effective amount of a FAAH inhibitor to said patient.

84. The method according to claim 83, wherein the dopaminergic agent is a dopamine replacement agent, a dopamine agonist, a dopamine uptake inhibitor or a monoamine oxidase inhibitor.

85. The method according to claim 84, wherein the dopamine replacement agent comprises melevodopa or L-3,4-dihydroxyphenylalanine (levodopa, L-DOPA), or an AADC enzyme inhibitor or both an AADC enzyme inhibitor and a COMT inhibitor.

86. The method according to claim 85, wherein the AADC enzyme inhibitor is carbidopa or benserazide and the COMT inhibitor, if present, is entacapone or tolcapone.

87. The method according to claim 84, wherein the dopamine agonist is bromocriptine (Parlodel®), pergolide (Permax®), pramipexole (Mirapex®), ropinirole (Requip®), rotigotine (Neupro®), cabergoline (Dostinex®), apomorphine (Apokyn®), lisuride (Dopergine®) or talipexole.

88. The method according to claim 84, wherein the dopamine uptake inhibitor is selected from BLS-602, BLS-605, SEP-226330, bupropion (Wellbutrin®, Zyban®), dexmethylphenidate (Focalin®) or methylphenidate (Ritalin®, Concerta®) and the monoamine oxidase inhibitor is selected from rasagiline (Azilect), selegiline or safinamide.

89. The method according to claim 83, wherein said DDS is characterized by punding, stereotypies, hobbyism or walkabouts, or wherein said ICD is selected from pathological gambling, an eating disorder, compulsive shopping or hypersexuality, or wherein said sleep disorder is selected from insomnia, restlessness, vivid dreaming, or nightmares.

90. The method according to claim 83, wherein said patient is a human.

91. The method according to claim 83, wherein said FAAH inhibitor is a compound of Formula A or a pharmaceutically acceptable salt thereof: 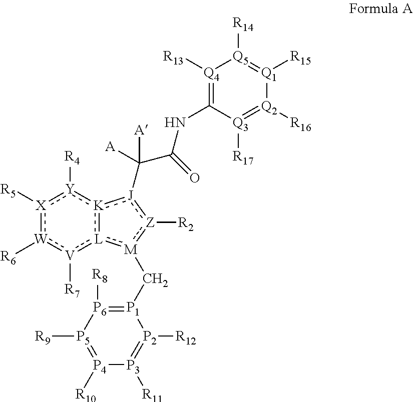
wherein:
V, W, X, Y, Z, J, K, L are C and M is N;
Q₁ is N and Q₂, Q₃, Q₄ and Q₅ are C;
a) P₁, P₂, P₃, P₄, P₅ and P₆ are C; or b) one of P₁, P₂, P₃, P₄, P₅ and P₆ is N and the rest are C;
A and A′ taken together are ═O; [symbol]
indicates a double or single bond;
R₂ is hydroxyl, an optionally independently substituted C1-C3 alkyl, an optionally independently substituted C₁-C₂ alkoxy or an optionally independently substituted cyclopropyl;
each of R₄, R₅, R₆ and R₇ is independently: H, a halogen, —NO₂, —CN, —C(O)OH, hydroxyl, an optionally independently substituted C1-C5 alkyl, an optionally independently substituted C2-C5 alkenyl, an optionally independently substituted C2-C5 alkynl, an optionally independently substituted C1-C5 alkoxy, —C(O)NRₐR_{b}, or —NRₐR_{b}, wherein Rₐ and R_{b} are independently H, an optionally independently substituted C1-C6 alkyl, or an optionally independently substituted C3-C6 cycloalkyl;
each of R₈, R₉, R₁₀, R₁₁ and R₁₂, when bonded to C, is independently: H, a halogen, —NO₂, —CN, —C(O)OH, hydroxyl, an optionally independently substituted C1-C5 alkyl, an optionally independently substituted C2-C5 alkenyl, an optionally independently substituted C2-C5 alkynl, an optionally independently substituted C1-C5 alkoxy, —C(O)NRₐR_{b}, or —NRₐR_{b}, wherein Rₐ and R_{b} are independently H, an optionally independently substituted C1-C6 alkyl, or an optionally independently substituted C3-C6 cycloalkyl;
each of R₈, R₉, R₁₀ R₁₁ and R₁₂, when bonded to N, is missing;
R₁₄ is selected from H, a halogen, —NO₂, —CN, —C(O)OH, hydroxyl, an optionally independently substituted C1-C5 alkyl, an optionally independently substituted C2-C5 alkenyl, an optionally independently substituted C2-C5 alkynl, an optionally independently substituted C1-C5 alkoxy, —C(O)NRₐR_{b}, or —NRₐR_{b}, wherein Rₐ and R_{b} are independently H, an optionally independently substituted C1-C6 alkyl, or an optionally independently substituted C3-C6 cycloalkyl;
R₁₆ is selected from H, a halogen, —NO₂, —CN, —C(O)OH, hydroxyl, an optionally independently substituted C1-C5 alkyl, an optionally independently substituted C2-C5 alkenyl, an optionally independently substituted C2-C5 alkynl, an optionally independently substituted C1-C5 alkoxy, —C(O)NRₐR_{b}, or —NRₐR_{b}, wherein Rₐ and R_{b} are independently H, optionally independently substituted C1-C6 alkyl, or an optionally independently substituted C3-C6 cycloalkyl;
R₁₅ is missing;
R₁₃ is selected from H, a halogen, —NO₂, —CN, —C(O)OH, hydroxyl, an optionally independently substituted C1-C5 alkyl, an optionally independently substituted C2-C5 alkenyl, an optionally independently substituted C2-C5 alkynl, an optionally independently substituted C1-C5 alkoxy, —C(O)NRₐR_{b}, or —NRₐR_{b}, wherein Rₐ and R_{b} are independently H, optionally independently substituted C1-C6 alkyl, or an optionally independently substituted C3-C6 cycloalkyl; and
R₁₇ is selected from H, a halogen, —NO₂, —CN, —C(O)OH, hydroxyl, an optionally independently substituted C1-C5 alkyl, an optionally independently substituted C2-C5 alkenyl, an optionally independently substituted C2-C5 alkynl, an optionally independently substituted C1-C5 alkoxy, —C(O)NRₐR_{b}, or —NRₐR_{b}, wherein Rₐ and R_{b} are independently H, optionally independently substituted C1-C6 alkyl, or an optionally independently substituted C3-C6 cycloalkyl.

92. The method of claim 91, wherein one of P₁, P₂, P₃, P₄, P₅ and P₆ is N and the rest are C.

93. The method of claim 91, wherein P₁, P₂, P₃, P₄, Pₛ and P₆ are C.

94. The method of claim 91, wherein R₂ is a C1-C3 alkyl or cyclopropyl.

95. The method of claim 94, wherein R₂ is methyl.

96. The method of claim 91, wherein one or two of R₈, R₉, R₁₀, R₁₁ and R₁₂ are halogen and the rest are H.

97. The method of claim 96, wherein one or two of R₈, R₉, R₁₀, R₁₁ and R₁₂ are Cl or F and the rest are H.

98. The method of claim 96, wherein R₁₀ is halogen.

99. The method of claim 96, wherein one of R₈ and R₁₂ is halogen and the other is H.

100. The method of claim 96, wherein R₁₀ is Cl or F and R₈, R₉, R₁₁ and R₁₂ are H.

101. The method of claim 96, wherein R₁₀ is Cl or F, R₈ is Cl or F; and R₉, R₁₁ and R₁₂ are H.

102. The method of claim 91, wherein R₄ and R₇ are independently H.

103. The method of claim 91, wherein R₆ is H.

104. The method of claim 102 wherein R₅ is selected from ethoxy, methoxy, ethyl, methyl, halogen and H.

105. The method of claim 104, wherein R₅ is selected from methoxy, methyl and H.

106. The method of claim 91, wherein R₁₆ is halogen or an optionally independently substituted methoxy and both R₁₃ and R₁₇ are H.

107. The method of claim 106, wherein R₁₆ is —OCH₃.

108. The method of claim 91, wherein the FAAH inhibitor is selected from the following list of compounds:
1-[(4-chlorophenyl)methyl]-N-(3-fluoro-4-pyridinyl)-5-methoxy-2-
methyl-α-oxo-1H-Indole-3-acetamide
1-[(2,4-difluorophenyl)methyl]-N-(2-methoxy-4-pyridinyl)-2,5-
dimethyl-α-oxo-1H-Indole-3-acetamide
N-(2-chloro-4-pyridinyl)-5-methoxy-1-[(4-methoxyphenyl)methyl]-2-
methyl-α-oxo-1H-Indole-3-acetamide
N-(2-chloro-4-pyridinyl)-1-[(4-fluorophenyl)methyl]-5-methoxy-2-
methyl-α-oxo-1H-Indole-3-acetamide
N-(2-chloro-4-pyridinyl)-1-[(2,4-dichlorophenyl)methyl]-5-methoxy-2-
methyl-α-oxo-1H-Indole-3-acetamide
5-methoxy-N-(2-methoxy-4-pyridinyl)-2-methyl-α-oxo-1-[[4-
(trifluoromethyl)phenyl]methyl]-1H-Indole-3-acetamide
5-methoxy-N-(2-methoxy-4-pyridinyl)-2-methyl-α-oxo-1-[[4-
(trifluoromethoxy)phenyl]methyl]-1H-Indole-3-acetamide
1-[(6-chloro-3-pyridinyl)methyl]-5-methoxy-N-(2-methoxy-4-pyridinyl)-
2-methyl-α-oxo-1H-Indole-3-acetamide
5-methoxy-N-(2-methoxy-4-pyridinyl)-2-methyl-1-[(4-methylphenyl)
methyl]-α-oxo-1H-Indole-3-acetamide
5-methoxy-1-[(4-methoxyphenyl)methyl]-N-(2-methoxy-4-pyridinyl)-
2-methyl-α-oxo-1H-Indole-3-acetamide
5-fluoro-1-[(4-fluorophenyl)methyl]-N-(2-methoxy-4-pyridinyl)-2-
methyl-α-oxo-1H-Indole-3-acetamide
5-chloro-1-[(4-fluorophenyl)methyl]-N-(2-methoxy-4-pyridinyl)-2-
methyl-α-oxo-1H-Indole-3-acetamide
5-chloro-1-[(4-chlorophenyl)methyl]-N-(2-methoxy-4-pyridinyl)-2-
methyl-α-oxo-1H-Indole-3-acetamide
1-[(4-fluorophenyl)methyl]-5-methoxy-N-(2-methoxy-4-pyridinyl)-
2-methyl-α-oxo-1H-Indole-3-acetamide
1-[(4-fluorophenyl)methyl]-N-(2-methoxy-4-pyridinyl)-2-methyl-α-
oxo-1H-Indole-3-acetamide
1-[(4-fluorophenyl)methyl]-N-(2-methoxy-4-pyridinyl)-2,5-
dimethyl-α-oxo-1H-Indole-3-acetamide
1-[(4-chlorophenyl)methyl]-5-methoxy-N-(2-methoxy-4-
pyridinyl)-2-methyl-α-oxo-1H-Indole-3-acetamide
1-[(4-chlorophenyl)methyl]-N-(2-fluoro-4-pyridinyl)-5-
methoxy-2-methyl-α-oxo-1H-Indole-3-acetamide
1-[(4-chlorophenyl)methyl]-N-(2-ethoxy-4-pyridinyl)-5-methoxy-2-
methyl-α-oxo-1H-Indole-3-acetamide
1-[(4-chlorophenyl)methyl]-5-fluoro-N-(2-methoxy-4-pyridinyl)-2-
methyl-α-oxo-1H-Indole-3-acetamide
1-[(4-chlorophenyl)methyl]-N-(2-chloro-4-pyridinyl)-5-fluoro-2-
methyl-α-oxo-1H-Indole-3-acetamide
1-[(4-chlorophenyl)methyl]-5-fluoro-2-methyl-α-oxo-N-4-pyridinyl-
1H-Indole-3-acetamide
1-[(4-chlorophenyl)methyl]-5-ethoxy-N-(2-methoxy-4-pyridinyl)-2-
methyl-α-oxo-1H-Indole-3-acetamide
1-[(4-chlorophenyl)methyl]-N-(2-chloro-4-pyridinyl)-5-ethoxy-2-
methyl-α-oxo-1H-Indole-3-acetamide
1-[(4-chlorophenyl)methyl]-N-(2-methoxy-4-pyridinyl)-2-methyl-α-oxo-
1H-Indole-3-acetamide
1-[(4-chlorophenyl)methyl]-N-(2-chloro-4-pyridinyl)-2-methyl-α-oxo-
1H-Indole-3-acetamide
1-[(4-chlorophenyl)methyl]-N-(2-methoxy-4-pyridinyl)-2,5-dimethyl-α-
oxo-1H-Indole-3-acetamide
1-[(4-chlorophenyl)methyl]-N-(2-chloro-4-pyridinyl)-2,5-dimethyl-α-
oxo-1H-Indole-3-acetamide
1-[(4-chloro-2-fluorophenyl)methyl]-5-methoxy-N-(2-methoxy-4-
pyridinyl)-2-methyl-α-oxo-1H-Indole-3-acetamide
1-[(4-chloro-2-fluorophenyl)methyl]-N-(2-methoxy-4-pyridinyl)-2-
methyl-α-oxo-1H-Indole-3-acetamide
1-[(4-chloro-2-fluorophenyl)methyl]-N-(2-methoxy-4-pyridinyl)-2,5-
dimethyl-α-oxo-1H-Indole-3-acetamide
1-[(3-chlorophenyl)methyl]-5-methoxy-N-(2-methoxy-4-pyridinyl)-
2-methyl-α-oxo-1H-Indole-3-acetamide
1-[(2-chlorophenyl)methyl]-5-methoxy-N-(2-methoxy-4-pyridinyl)-
2-methyl-α-oxo-1H-Indole-3-acetamide
1-[(2-chloro-4-fluorophenyl)methyl]-5-methoxy-N-(2-methoxy-
4-pyridinyl)-2-methyl-α-oxo-1H-Indole-3-acetamide
1-[(2-chloro-4-fluorophenyl)methyl]-N-(2-methoxy-4-pyridinyl)-
2-methyl-α-oxo-1H-Indole-3-acetamide
1-[(2-chloro-4-fluorophenyl)methyl]-N-(2-methoxy-4-pyridinyl)-
2,5-dimethyl-α-oxo-1H-Indole-3-acetamide
1-[(2,4-difluorophenyl)methyl]-5-methoxy-N-(2-methoxy-4-pyridinyl)-
2-methyl-α-oxo-1H-Indole-3-acetamide
1-[(2,4-difluorophenyl)methyl]-N-(2-methoxy-4-pyridinyl)-2-
methyl-α-oxo-1H-Indole-3-acetamide
1-[(2,4-dichlorophenyl)methyl]-5-methoxy-N-(2-methoxy-4-pyridinyl)-
2-methyl-α-oxo-1H-Indole-3-acetamide
1-[(2,4-dichlorophenyl)methyl]-5-fluoro-N-(2-methoxy-4-pyridinyl)-
2-methyl-α-oxo-1H-Indole-3-acetamide
1-[(2,4-dichlorophenyl)methyl]-N-(2-methoxy-4-pyridinyl)-2-
methyl-α-oxo-1H-Indole-3-acetamide
1-[(2,4-dichlorophenyl)methyl]-N-(2-methoxy-4-pyridinyl)-
2,5-dimethyl-α-oxo-1H-Indole-3-acetamide
5-methoxy-N-(2-methoxy-4-pyridinyl)-2-methyl-α-oxo-1-
(phenylmethyl)-1H-Indole-3-acetamide
N-(2-methoxy-4-pyridinyl)-2-methyl-α-oxo-1-(phenylmethyl)-1H-
Indole-3-acetamide
N-(2-methoxy-4-pyridinyl)-2,5-dimethyl-α-oxo-1-(phenylmethyl)-1H-
Indole-3-acetamide
5-chloro-1-[(4-chlorophenyl)methyl]-N-(2-chloro-4-pyridinyl)-2-
methyl-α-oxo-1H-Indole-3-acetamide
5-chloro-1-[(4-chlorophenyl)methyl]-2-methyl-α-oxo-N-4-pyridinyl-
1H-Indole-3-acetamide
1-[(4-chlorophenyl)methyl]-N-(2-chloro-4-pyridinyl)-5-methoxy-2-
methyl-α-oxo-1H-Indole-3-acetamide
1-[(4-chlorophenyl)methyl]-5-hydroxy-2-methyl-α-oxo-N-4-pyridinyl-
1H-Indole-3-acetamide
1-[(4-chlorophenyl)methyl]-5-ethoxy-2-methyl-α-oxo-N-4-pyridinyl-
1H-Indole-3-acetamide
1-[(4-chlorophenyl)methyl]-2-methyl-α-oxo-N-4-pyridinyl-1H-Indole-
3-acetamide
1-[(4-chlorophenyl)methyl]-5-methyl-2-(1-methylethyl)-α-oxo-N-4-
pyridinyl-1H-Indole-3-acetamide
1-[(4-chlorophenyl)methyl]-2,5-dimethyl-α-oxo-N-4-pyridinyl-1H-
Indole-3-acetamide
1-[(2,4-dichlorophenyl)methyl]-5-methoxy-2-methyl-α-oxo-N-4-
pyridinyl-1H-Indole-3-acetamide
1-[(4-chlorophenyl)methyl]-5-methoxy-2-methyl-α-oxo-N-4-pyridinyl-
1H-Indole-3-acetamide

109. A pharmaceutical composition comprising a dopaminergic agent and a FAAH inhibitor.

110. The pharmaceutical composition according to claim 109, wherein said dopaminergic agent is a dopamine replacement agent.

111. The pharmaceutical composition according to claim 110, wherein the dopamine replacement agent comprises melevodopa or L-3,4-dihydroxypheylalanine (levodopamine; L-DOPA).

112. The pharmaceutical composition according to claim 111, wherein the dopamine replacement agent further comprises an AADC enzyme inhibitor or an AADC inhibitor and a COMT inhibitor.

113. The pharmaceutical composition according to claim 112, wherein the AADC enzyme inhibitor is carbidopa or benserazide and the COMT inhibitor, if present, is entacapone or tolcapone.

114. The pharmaceutical composition according to claim 109, wherein the dopaminergic agent is a dopamine agonist.

115. The pharmaceutical composition according to claim 114, wherein the dopamine agonist is bromocriptine (Parlodel®), pergolide (Permax®), pramipexole (Mirapex®), ropinirole (Requip®), rotigotine (Neupro®), cabergoline (Dostinex®), apomorphine (Apokyn®), lisuride (Dopergine®) or talipexole.

116. The pharmaceutical composition according to claim 109, wherein the dopaminergic agent is a dopamine uptake inhibitor.

117. The pharmaceutical composition according to claim 116, wherein the dopamine uptake inhibitor is BLS-602/BLS-605 or SEP-226330.

118. The pharmaceutical composition according to claim 109, wherein said FAAH inhibitor is a compound of Formula A or a pharmaceutically acceptable salt thereof: 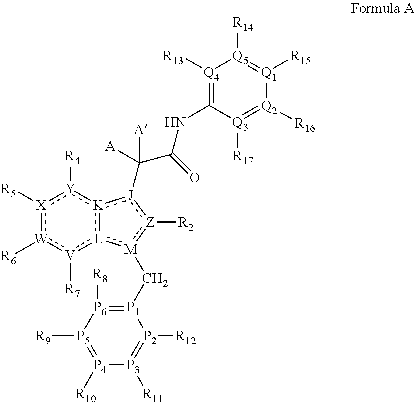
wherein:
V, W, X, Y, Z, J, K, L are C and M is N;
Q₁ is N and Q₂, Q₃, Q₄ and Q₅ are C;
b) P₁, P₂, P₃, P₄, P₅ and P₆ are C; or b) one of P₁, P₂, P₃, P₄, P₅ and P₆ is N and the rest are C;
A and A′ taken together are ═O; [symbol]
indicates a double or single bond;
R₂ is hydroxyl, an optionally independently substituted C1-C3 alkyl, an optionally independently substituted C1-C2 alkoxy or an optionally independently substituted cyclopropyl;
each of R₄, R₅, R₆ and R₇ is independently: H, a halogen, —NO₂, —CN, —C(O)OH, hydroxyl, an optionally independently substituted C1-C5 alkyl, an optionally independently substituted C2-C5 alkenyl, an optionally independently substituted C2-C5 alkynl, an optionally independently substituted C1-C5 alkoxy, —C(O)NRₐR_{b}, or —NRₐR_{b}, wherein Rₐ and R_{b} are independently H, an optionally independently substituted C1-C6 alkyl, or an optionally independently substituted C3-C6 cycloalkyl;
each of R₈, R₉, R₁₀, R₁₁ and R₁₂, when bonded to C, is independently: H, a halogen, —NO₂, —CN, —C(O)OH, hydroxyl, an optionally independently substituted C1-C5 alkyl, an optionally independently substituted C2-C5 alkenyl, an optionally independently substituted C2-C5 alkynl, an optionally independently substituted C1-C5 alkoxy, —C(O)NRₐR_{b}, or —NRₐR_{b}, wherein Rₐ and R_{b} are independently H, an optionally independently substituted C1-C6 alkyl, or an optionally independently substituted C3-C6 cycloalkyl;
each of R₈, R₉, R₁₀ R₁₁ and R₁₂, when bonded to N, is missing;
R₁₄ is selected from H, a halogen, —NO₂, —CN, —C(O)OH, hydroxyl, an optionally independently substituted C1-C5 alkyl, an optionally independently substituted C2-C5 alkenyl, an optionally independently substituted C2-C5 alkynl, an optionally independently substituted C1-C5 alkoxy, —C(O)NRₐR_{b}, or —NRₐR_{b}, wherein Rₐ and R_{b} are independently H, an optionally independently substituted C1-C6 alkyl, or an optionally independently substituted C3-C6 cycloalkyl;
R₁₆ is selected from H, a halogen, —NO₂, —CN, —C(O)OH, hydroxyl, an optionally independently substituted C1-C5 alkyl, an optionally independently substituted C2-C5 alkenyl, an optionally independently substituted C2-C5 alkynl, an optionally independently substituted C1-C5 alkoxy, —C(O)NRₐR_{b}, or —NRₐR_{b}, wherein Rₐ and R_{b} are independently H, optionally independently substituted C1-C6 alkyl, or an optionally independently substituted C3-C6 cycloalkyl;
R₁₅ is missing;
R₁₃ is selected from H, a halogen, —NO₂, —CN, —C(O)OH, hydroxyl, an optionally independently substituted C1-C5 alkyl, an optionally independently substituted C2-C5 alkenyl, an optionally independently substituted C2-C5 alkynl, an optionally independently substituted C1-C5 alkoxy, —C(O)NRₐR_{b}, or —NRₐR_{b}, wherein Rₐ and R_{b} are independently H, optionally independently substituted C1-C6 alkyl, or an optionally independently substituted C3-C6 cycloalkyl; and
R₁₇ is selected from H, a halogen, —NO₂, —CN, —C(O)OH, hydroxyl, an optionally independently substituted C1-C5 alkyl, an optionally independently substituted C2-C5 alkenyl, an optionally independently substituted C2-C5 alkynl, an optionally independently substituted C1-C5 alkoxy, —C(O)NRₐR_{b}, or —NRₐR_{b}, wherein Rₐ and R_{b} are independently H, optionally independently substituted C1-C6 alkyl, or an optionally independently substituted C3-C6 cycloalkyl.
